(19) United States
(12) Patent Application Publication
(10) Pub. No.: US 2004/0180322 A1
(43) Pub. Date: Sep. 16, 2004

(54) Regional intestinal permeability model

(76) Inventors: George M. Grass, Tahoe City, CA (US); Glen D. Leesman, Hamilton, MT (US); Daniel A. Norris, San Diego, CA (US); Patrick J. Sinko, Lebanon, NJ (US); Jehangir Athwal, San Diego, CA (US); Carleton Sage, Cardiff-by-the Sea, CA (US); Troy Bremer, Dana Point, CA (US); Kevin Holme, San Diego, CA (US); Yong-Hee Lee, San Diego, CA (US); Kyoung Lee, Blue Bell, PA (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/332,999

(22) PCT Filed: Jul. 30, 2001
(86) PCT No.: PCT/US01/23762

Related U.S. Application Data
(60) Provisional application No. 60/221,548, filed on Jul. 28, 2000.
(60) Provisional application No. 60/267,435, filed on Feb. 9, 2001.
(60) Provisional application No. 60/277,952, filed on Mar. 23, 2001.
(60) Provisional application No. 60/288,466, filed on May 4, 2001.

(51) Int. Cl. C12Q 1/00; G06F 19/00; G01N 33/48; G01N 33/50

(52) U.S. Cl. 435/4; 702/19

(57) ABSTRACT

Permeability models and methods for creating the models are disclosed. The models include receiving as an input in vitro permeability and structure data for a particular compound. Then the data is mapped to at least one permeability. In some models the data is mapped to a plurality of permeabilities, each associated with a specific region in a mammalian GI tract. Some models may take into consideration solubility, permeability and at least one molecular descriptor associated with the compound of interest.

This application claims the benefit of U.S. Provisional Application No. 60/221,548 filed Jul. 28, 2000, entitled PHARMACOKINETIC-BASED DRUG DESIGN TOOL AND METHOD; 60/267,435 filed Feb. 9, 2001 entitled SYSTEM AND METHOD FOR PREDICTING ADME CHARACTERISTICS OF A COMPOUND BASED ON ITS STRUCTURE; 60/277,952 filed Mar. 23, 2001, entitled CACO-2 PERMEABILITY MODEL; and 60/288,466 filed May 4, 2001 entitled CACO-2 PERMEABILITY MODEL.

### BACKGROUND OF THE INVENTION

1. Field of the Invention

This invention relates generally to chemical compound permeability models in mammals and more particularly, to regional intestinal permeability models that predict permeability from CACO-2 or other in vitro assay permeability data.

2. Description of the Related Art

Permeability models for modeling the permeability of a specific compound at a specific location in, for example, the small intestine are known in the art. Additionally, models of permeability for specific compounds tested in CACO-2 cell lines have been also developed. Typically, these models are for specific compounds and only map to a specific region in the intestine or in the GI tract. These models, when expanded to, include a plurality of compounds and/or a plurality of regions in the GI tract have insufficient accuracy and hence usability in modeling the permeability and absorption of a large number of compounds in the mammalian GI tract.

Consequently, a more robust and/or accurate model is needed that can efficiently utilize in vitro permeability data and map this data to at least one permeability while maintaining a high degree accuracy over a plurality of compounds. A more robust and/or accurate model is also needed that can efficiently utilize in vitro permeability data and map this data to a plurality regions in the GI tract while maintaining a high degree accuracy over a plurality of compounds.

### SUMMARY OF THE INVENTION

A regional intestinal permeability model, the model includes receiving as an input CACO-2 or other in vitro permeability and molecular structural data for a particular compound. Then the data is mapped to at least one permeability. In some embodiments the data may be mapped to plurality of permeability coefficients for specific regions in a mammalian GI tract. The mappings may take into consideration such factors as the solubility, permeability and molecular descriptors associated with the compound of interest.

### BRIEF DESCRIPTION OF THE SEVERAL VIEWS OF THE DRAWINGS

The accompanying drawings incorporated in and forming part of the specification illustrate several aspects of the present invention, and together with the description explain the principles of the invention. In the drawings:

FIGS. 1-37 illustrate and describe the present invention.

FIG. 38 is a block diagram of a system for predicting the ADME/Tox properties of a candidate drug;

FIG. 39 is a flow chart of the method for developing a model that will predict the ADME/Tox properties of a candidate drug; and for predicting the ADME/Tox properties of a candidate drug.

FIGS. 40-82 are individual showings of particular points pertinent and important to the present invention and illustrate specific examples of an embodiment of the invention aimed at predicting human ADME data.

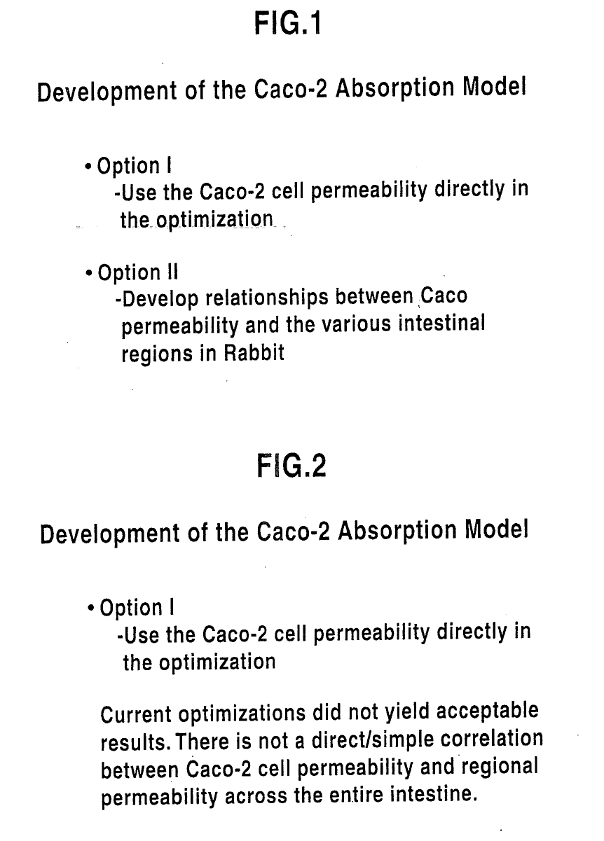

Reference will now be made in detail to the present preferred embodiment of the invention, examples of which are illustrated in the accompanying drawings.

### DETAILED DESCRIPTION OF INVENTION

1. Definitions

The following bolded terms are used throughout this document with the following associated meanings:

Absorption: Transfer of a compound across a physiological barrier as a function of time and initial concentration. Amount or concentration of the compound on the external and/or internal side of the barrier is a function of transfer rate and extent, and may range from zero to unity.

Affine Regression: Linearly combining input data to approximate output data. This is essentially a linear regression that does not require the regression to go through zero.

Bioavailability: Fraction of an administered dose of a compound that reaches the sampling site and/or site of action. May range from zero to unity. Can be assessed as a function of time.

Boosting: A general method which attempts to increase the accuracy of a learning algorithm.

Compound: Chemical entity. Could be a drug, a gene, etc.

Computer Readable Medium: Medium for storing, retrieving and/or manipulating information using a computer. Includes optical, digital, magnetic mediums and the like; examples include portable computer diskette, CD-ROMs, hard drive on computer etc. Includes remote access mediums; examples include internet or intranet systems. Permits temporary or permanent data storage, access and manipulation.

Cross Validation: Used to estimate the generalization error. This method is based on resampling the data set, using randomly (or otherwise chosen) samples of the training set as test sets.

Data: Experimentally collected and/or predicted variables. May include dependent and independent variables.

Input Data: Data which is used as an input in the training or execution of a model. Could be either experimentally determined or calculated.

Target Data: Data for which a model is generated. Could be either experimentally determined or predicted.

Test Data: Experimentally determined data.

Descriptor: An element of the input data.

Committee Machine: A model that is comprised of a number of submodels such that the knowledge acquired by the submodels is fused to provide a superior answer to any of the independent submodels.

Regression/Classification: Methods for mapping the input data to the target data. Regression refers to the methods applicable to forming a continuous prediction of the target data, while classification (or in general pattern recognition) refers the methods applicable to separating the target data into groups or classes. The specific methods for performing the regression or classification include where appropriate: Affine or Linear Regressions, Kernel based methods, Artificial Neural Networks, Finite State Machines using appropriate methods to interpret probability distributions such as Maximum A Posteriori, Nearest Neighbor Methods, Decision Trees, Fisher's Discriminate Analysis.

Mapping: The process of relating the input data space to the target data space, which is accomplished by regression/classification and produces a model that predicts or classifies the target data. A model maps a set of input values to a set of target values.

Feature Selection Methods: The method of selecting desirable descriptors from the input data to enable the prediction or classification of the target data. This is typically accomplished by forward selection, backward selection, branch and bound selection, genetic algorithmic selection, or evolutionary selection.

ADME: Properties of absorption, distribution, metabolism, and excretion and encompasses other measures related to absorption, distribution, metabolism, and excretion. For example, heptocyte turnover or Caco-2 effective permeability.

Dissolution: Process by which a compound becomes dissolved in a solvent.

Fisher's Discriminate Analysis: A linear method which reduces the input data dimension by appropriately weighting the descriptors in order to best aid the linear separation and thus classification of target data.

Genetic Algorithms: Based upon the natural selection mechanism. A population of models undergo mutations and only those which perform the best contribute to the subsequent population Of models.

Input/Output System: Provides a user interface between the user and a computer system.

Kernel Representations: Variations of classical linear techniques employing a Mercer's Kernel or variations to incorporate specifically defined classes of nonlinearity. These include Fisher's Discriminate Analysis and principal component analysis. Kernel Representations as used by the present invention are described in the article, “Fisher Discriminate Analysis with Kernels,” Sebastian Mika, Gunnar Ratsch, Jason Weston, Bernhard Scholkopf, and Klaus-Robert Muller, GMD FIRST, Rudower Chaussee 5, 12489 Berlin, Germany, © IEEE 1999 (0-7803-5673-X/99), and in the article, “GA-based Kernel Optimization for Pattern Recognition: Theory for EHW Application,” Moritoshi Yasunaga, Taro Nakamura, Ikuo Yoshihara, and Jung Kim, IEEE© 2000 (0-7803-6375-2/00), which are both hereby incorporated herein by reference.

Model: a mathematical description of the relationship (correspondence) between at least one input value and at least one target value. A model may be generated or represented by any know means (e.g. Linear regression, Non-Linear Regression, Classification, Lookup Table, Transformation, etc.). A model may also be considered to represent a map that moves input space into target space.

Metabolism: Conversion of a compound (the parent compound) into one or more different chemical entities (metabolites).

Artificial neural networks: A parallel and distributed system made up of the interconnection of simple processing units. Artificial neural networks as used in the present invention are described in detail in the book entitled, “Neural networks, A Comprehensive Foundation,” Second Edition, Simon Haykin, McMaster University, Hamilton, Ontario, Canada, published by Prentice Hall© 1999, which is hereby incorporated herein by reference.

Permeability: Ability of a barrier to permit passage of a substance or the ability of a substance to pass through a barrier. Refers to the concentration-dependent or concentration-independent rate of transport (flux), and collectively reflects the effects of characteristics such as molecular size, charge, partition coefficient and stability of a compound on transport. Permeability is substance and/or barrier specific. A measure of the movement of a compound across a membrane. Permeability may also be referred to as: Effective Permeability, Apparent Permeability, Permeability Coefficient, Permeability Rate. Permeability can be mathematically represented as: J=P*DELTA(Concentation), where J=flux, P=Permeability coefficient, and DELTA(Concentraction)=the change with time of the concentration of a compound. The membranes for which permeability values are determined, may be from any relevant in vitro, in situ, or ex vivo source (e.g. artificial synthetic membrane, immortal cell lines, animal intestinal tissue, etc.)

Physiologic Pharmacokinetic Model: Mathematical model describing movement and disposition of a compound in the body or an anatomical part of the body based on pharmacokinetics and physiology.

Principal Component Analysis: A type of non-directed data compression which uses a linear combination of features to produce a lower dimension representation of the data. An example of principal component analysis as applicable to use in the present invention is described in the article, “Nonlinear Component Analysis as a Kernel Eigenvalue Problem,” Bernhard Scholkopt, Neural Computation, Vol. 10, Issue 5, pp. 1299-1319, 1998, MIT Press., and is hereby incorporated herein by reference.

Simulation Engine: Computer-implemented instrument that simulates behavior of a system using an approximate mathematical model of the system. Combines mathematical model with user input variables to simulate or predict how the system behaves. May include system control components such as control statements (e.g., logic components and discrete objects).

Solubility: Property of being soluble; relative capability of being dissolved.

Support Vector Machines: Method which regresses/classifies by projecting input data into a higher dimensional space. Examples of Support Vector machines and methods as applicable to the present invention are described in the article, “Support Vector Methods in Learning and Feature Extraction,” Berhard Scholkopf, Alex Smola, Klaus-Robert Muller, Chris Burges, Vladimir Vapnik, Special issue with selected papers of ACNN'98, Australian Journal of Intelligent Information Processing Systems, 5 (1), 3-9), and in the article, “Distinctive Feature Detection using Support Vector Machines,” Partha Niyogi, chris Burges, and Padma Ramesh, Bell Labs, Lucent Technologies, USA, IEEE© 1999 (0-7803-5041-3/99), which are both hereby incorporated herein by reference.

2. Preferred Embodiments

This specification specifically incorporates by reference U.S. Provisional Application Serial No. ______, entitled System and Method for Predicting ADME Characteristics of a Compound Based on its Structure, filed Feb. 9, 2001. This provisional application describes in detail an exemplary model that could be utilized to produce the specific regional intestinal permeability model described below.

This specification also incorporates by reference the following U.S. patent application Ser. No. 09/320,372, filed May 26, 1999; Ser. No. 09/320,270, filed May 26, 1999; Ser. No. 09/320,371, filed May 26, 1999; Ser. No. 09/320,545, filed May 26, 1999; Ser. No. 09/320,544, filed May 26, 1999; and Ser. No. 09/320,069, filed May 26, 1999. This application also incorporates by reference PCT Application Serial Nos. PCT/US99/21001, filed on Sep. 14, 1999 and PCT/US99/21151, filed Sep. 14, 1999. The above U.S. and PCT applications disclose the details of a compound absorption model that could utilize the regional intestinal permeability model to enable faster/more efficient screening of compounds. Additionally, the absorption model described in these applications could be enhanced by employing the output of the regional intestinal permeability model as an input to the absorption model.

The ability to map the in vitro permeability data to intestinal permeability for each section of the GI tract of interest will enable the aforementioned absorption model to be utilized in an earlier stage in the drug development process based on in vitro assay results and still provide reliable results.

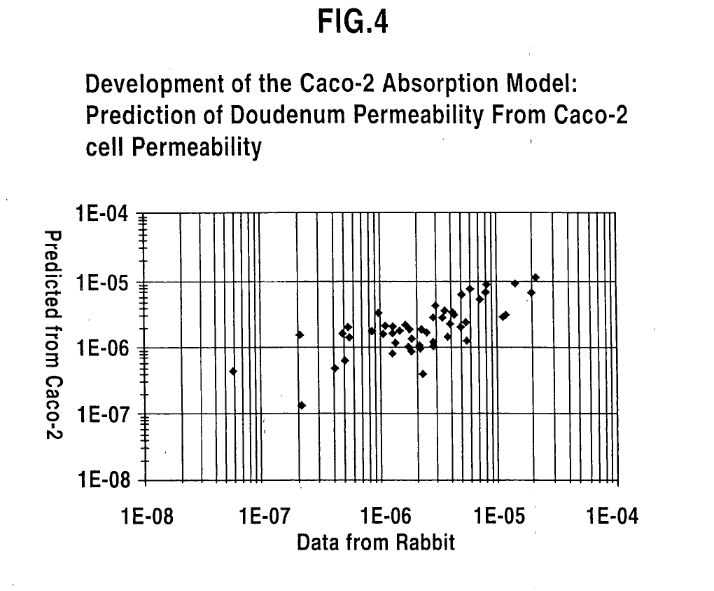

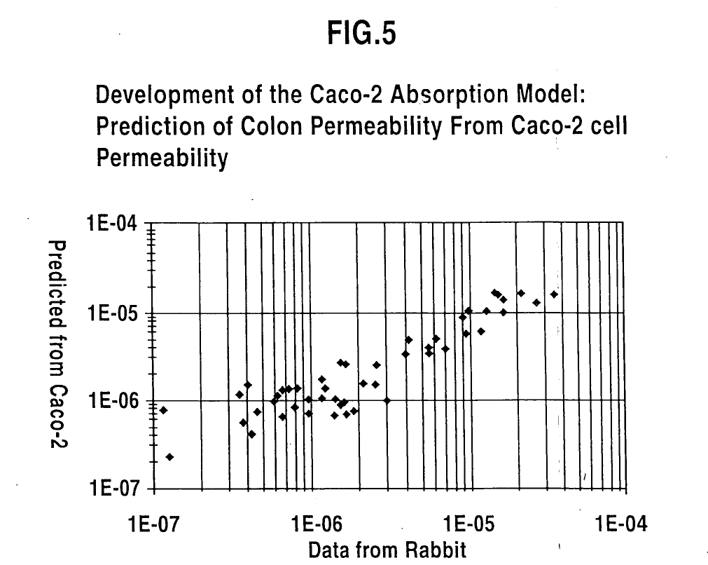

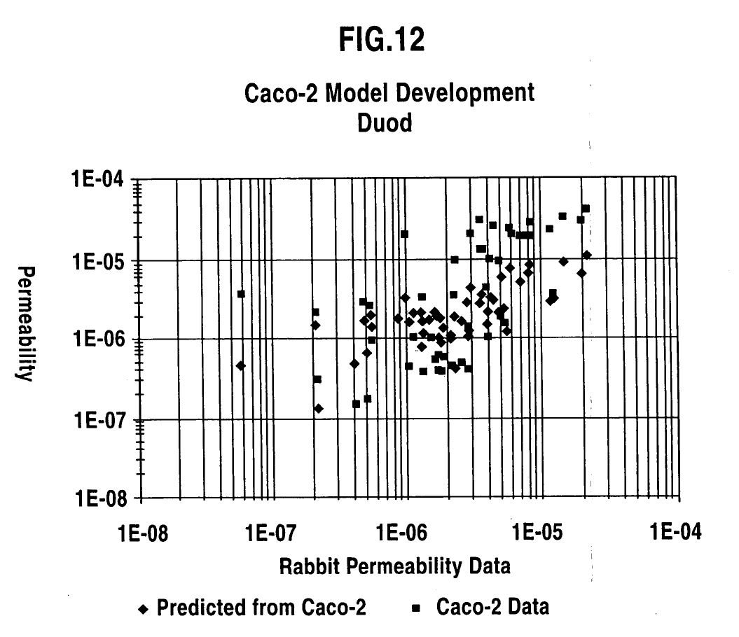

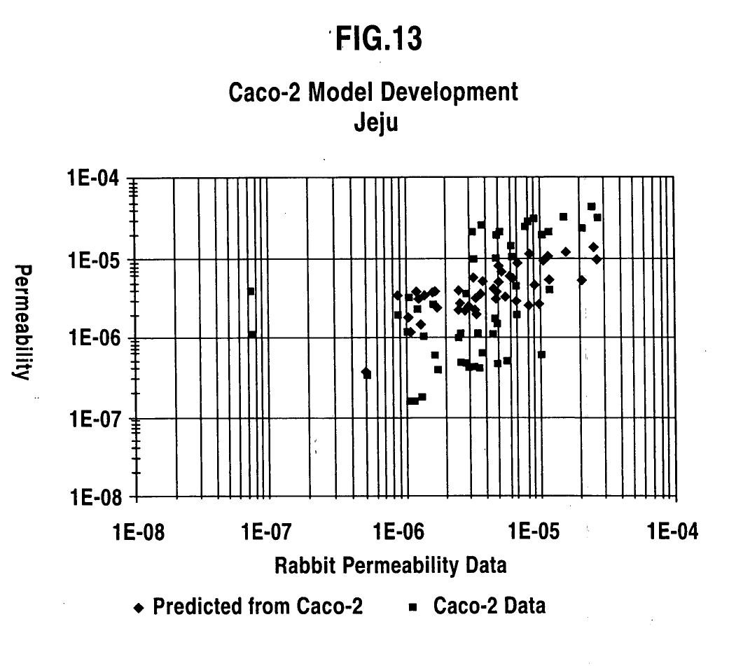

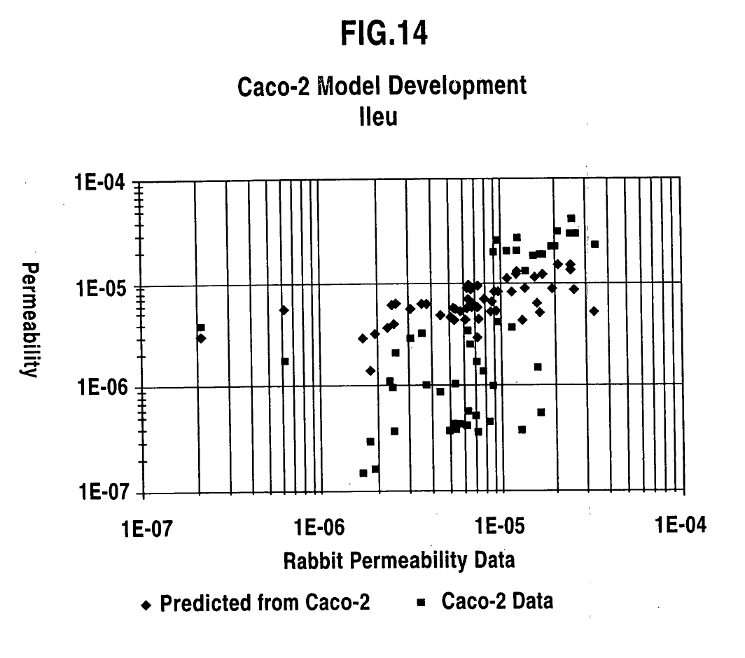

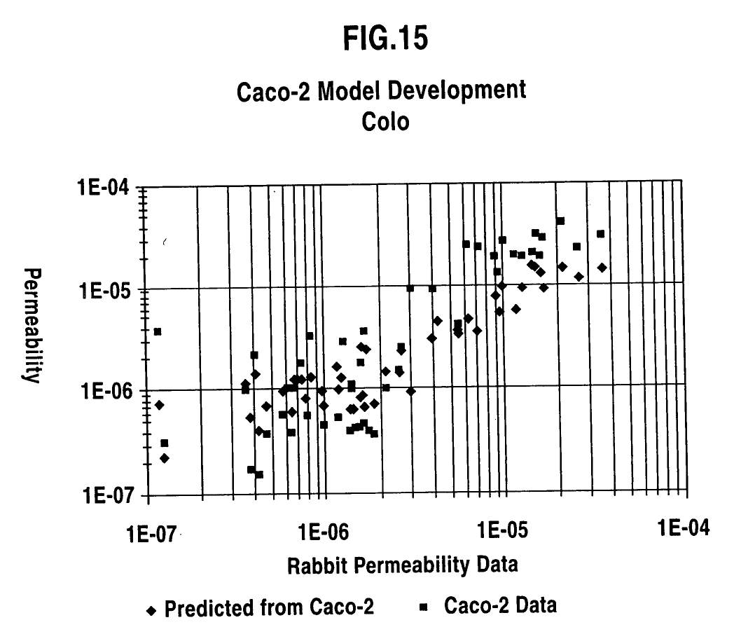

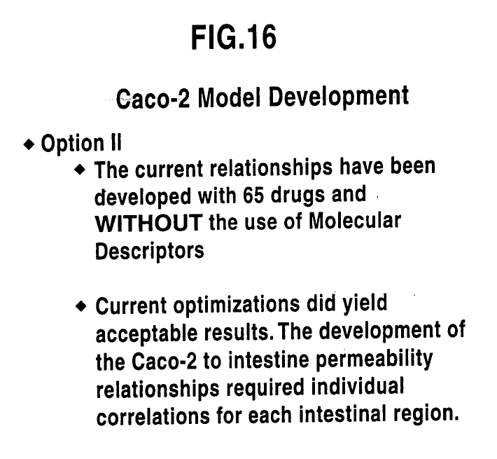

The basic approach to developing the regional intestinal permeability model involved developing relationships between CACO-2 cell permeability in the various intestinal regions in mammalian species. Due to the availability of data, the various intestinal regions in the rabbit were used in developing the model. The initial model was developed using the results of assaying a large and chemically diverse set of compounds for permeability in CACO-2 cells and in the four intestinal regions of the rabbit (colon, duodenum, ileum, and jejunum). This model utilized the non-linear regression techniques discussed in the U.S. Provisional Application, incorporated by reference above, to develop the first embodiment of the model. FIG. 4 illustrates the prediction of duodenum permeability using CACO-2 cell permeability as an input compared to the actual permeability assay data from the rabbit. FIG. 5 provides a similar illustration for the prediction for the colon in a rabbit. FIGS. 12-15 illustrate a second comparison of the predicted permeability from the regional intestinal permeability model to the rabbit permeability data for the duodenum ileum, and colon.

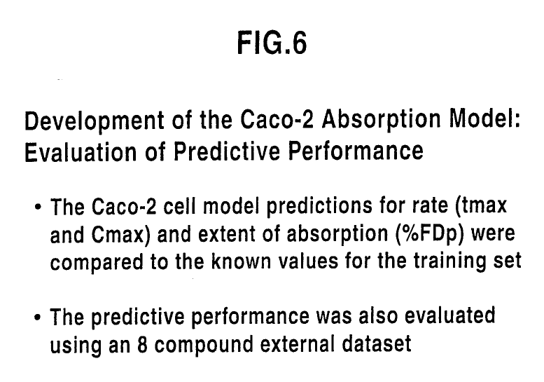

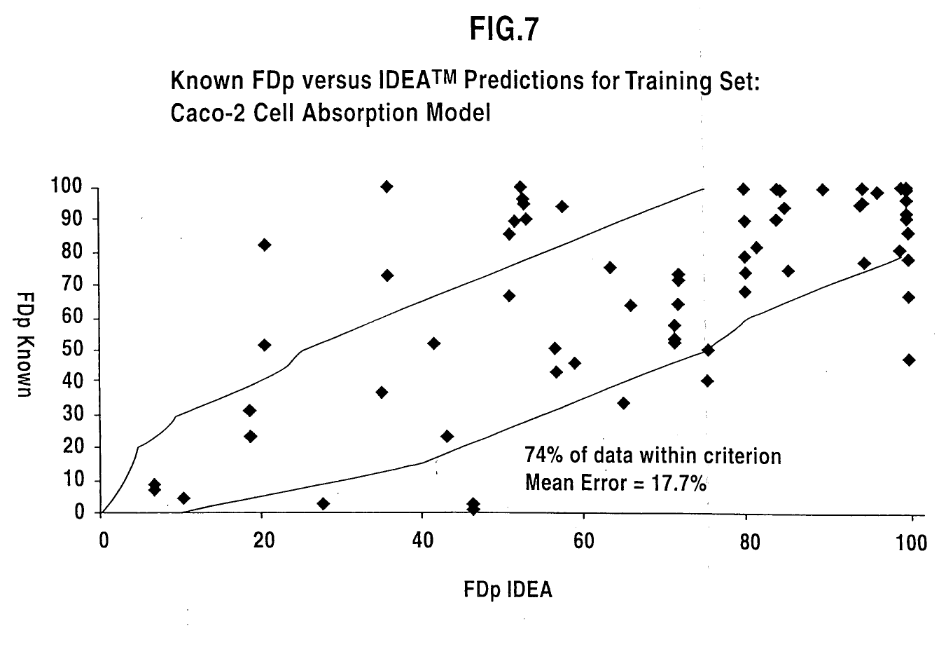

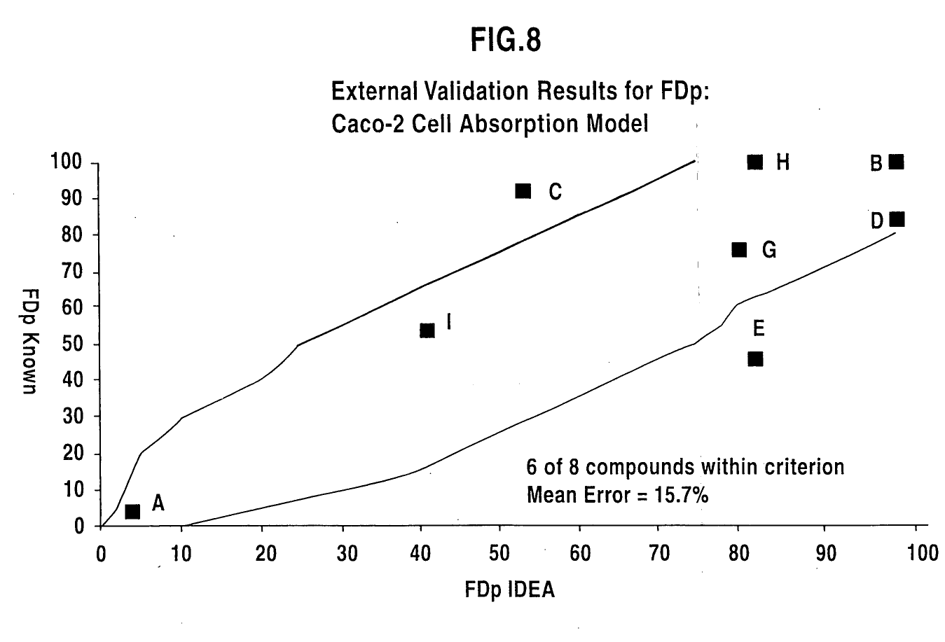

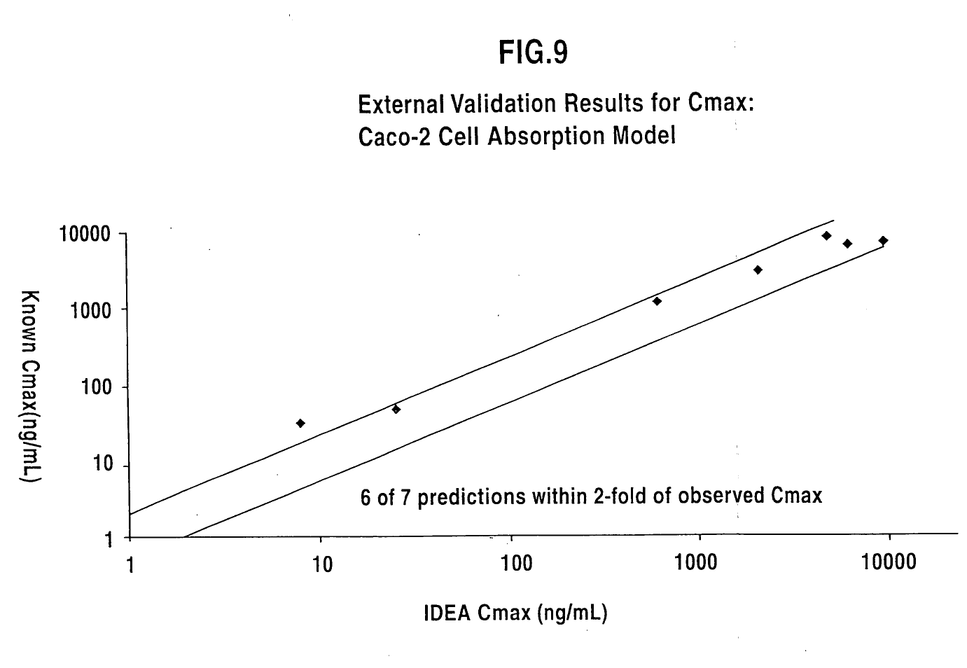

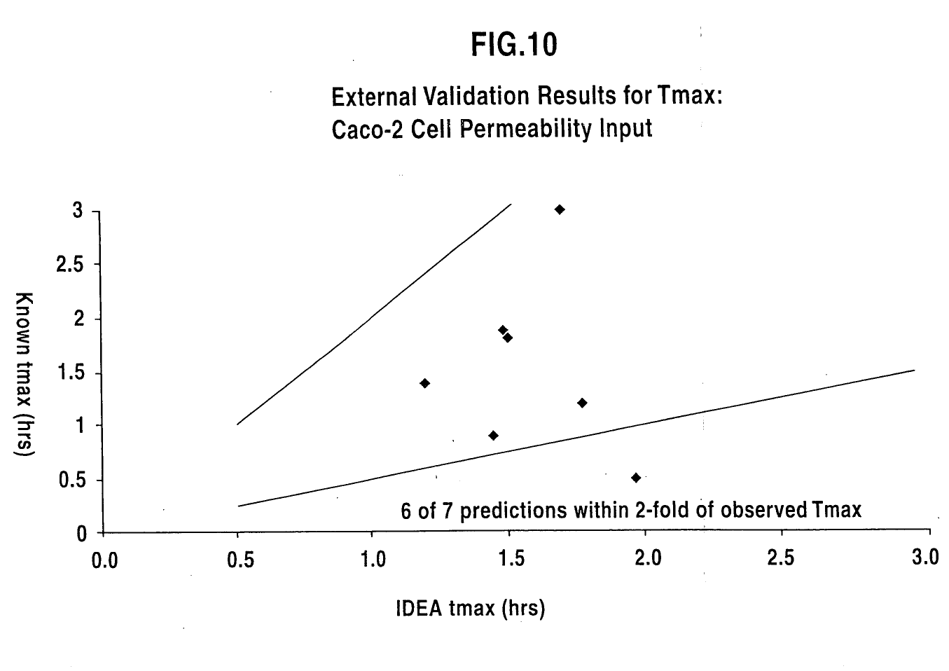

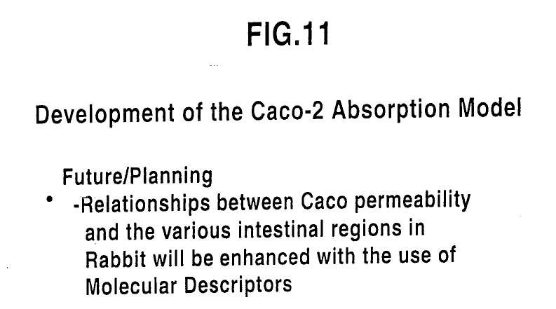

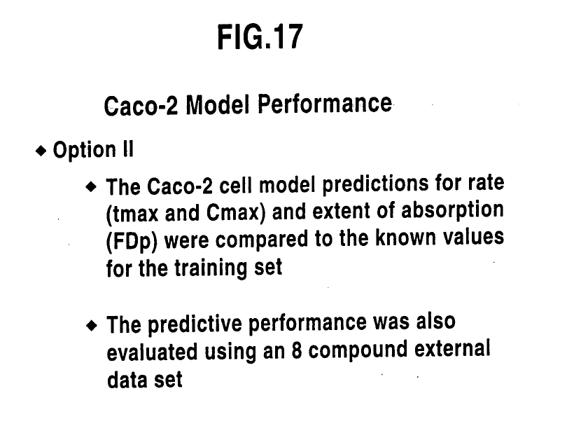

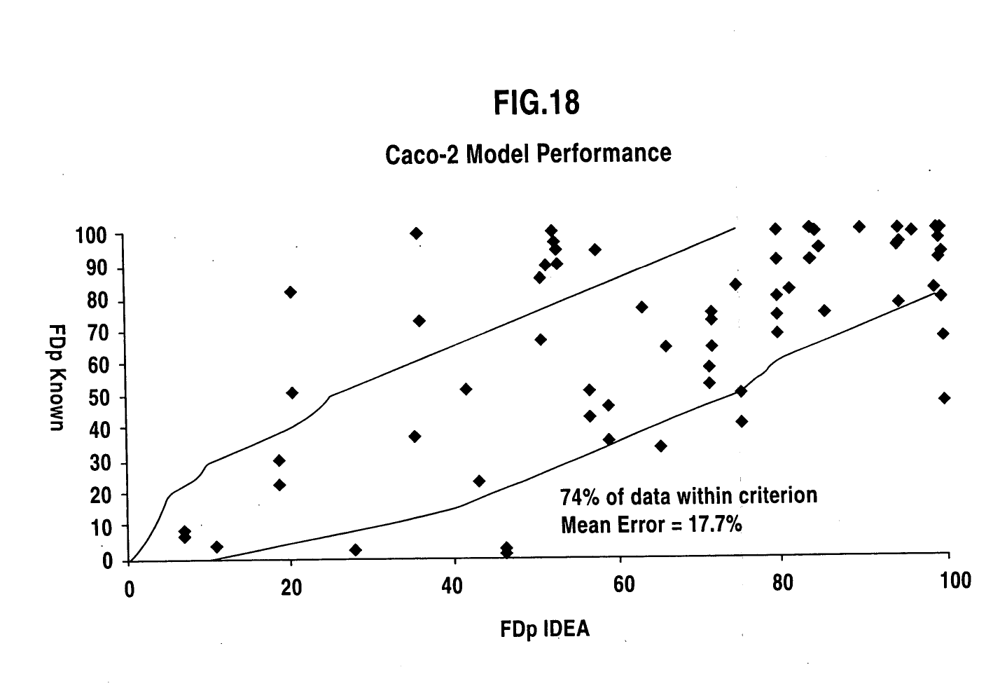

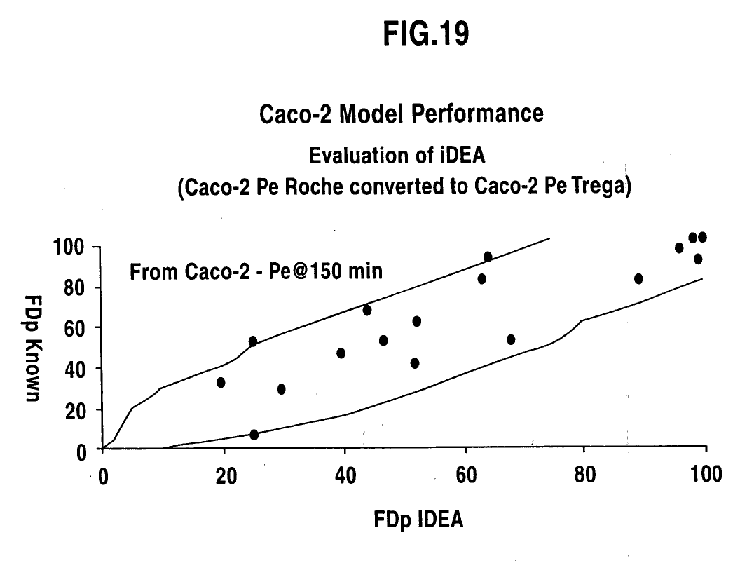

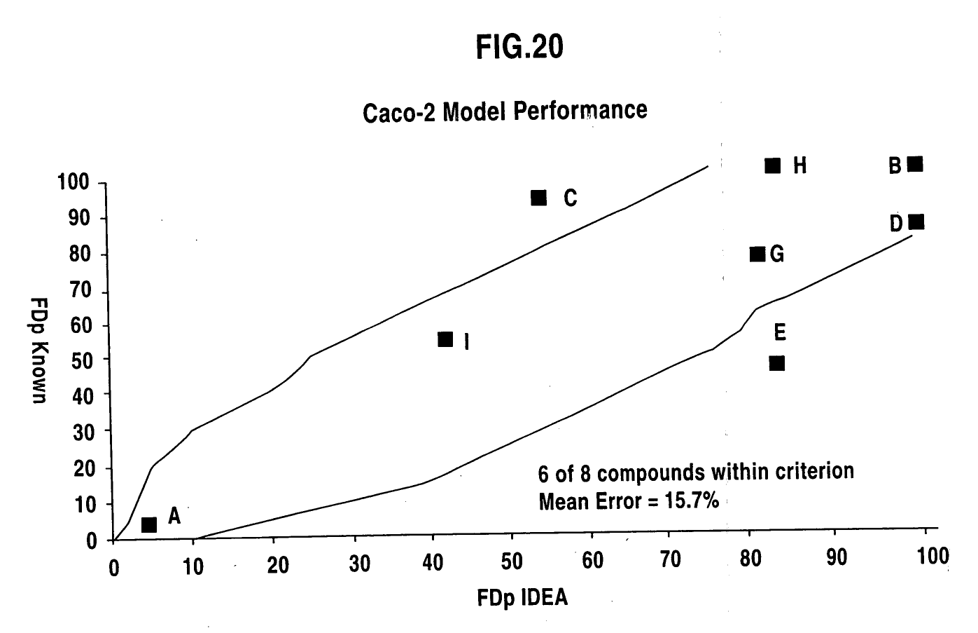

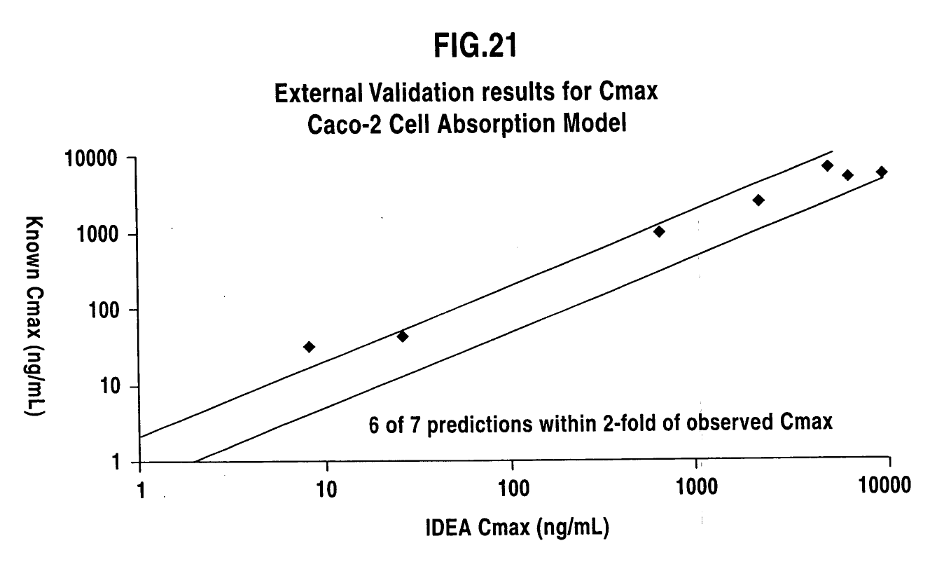

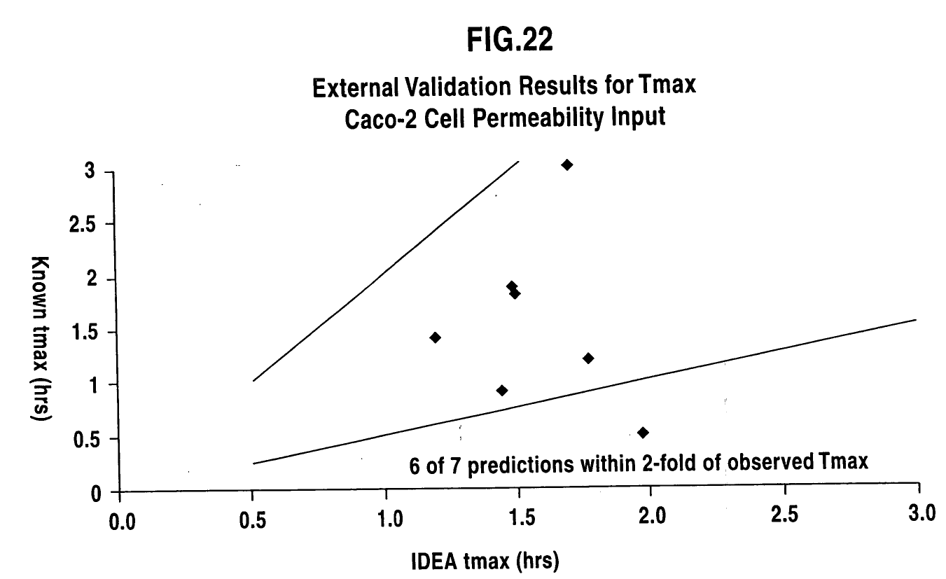

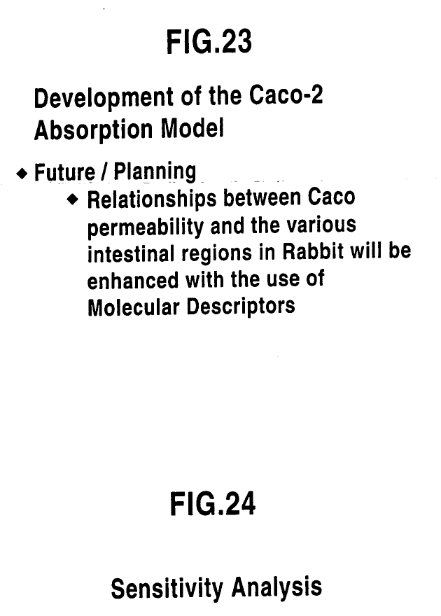

FIGS. 6-10 illustrate the performance of an absorption model based on the model disclosed in the U.S. and PCT applications using the regional intestinal permeability model to provide permeability based on in vitro permeability data. FIGS. 17-23 illustrate the additional performance data.

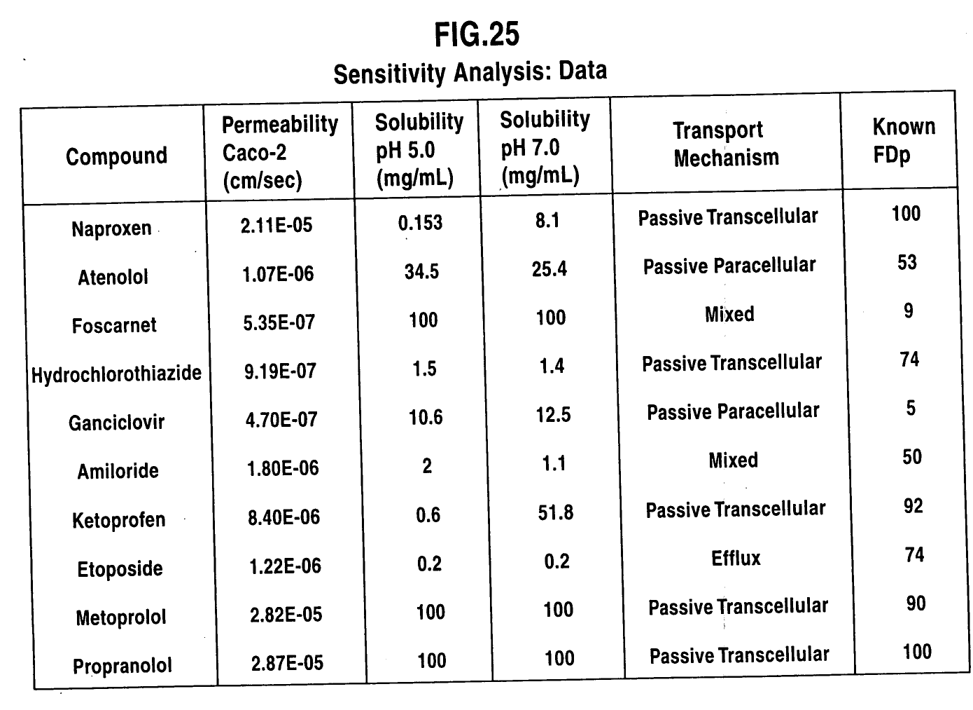

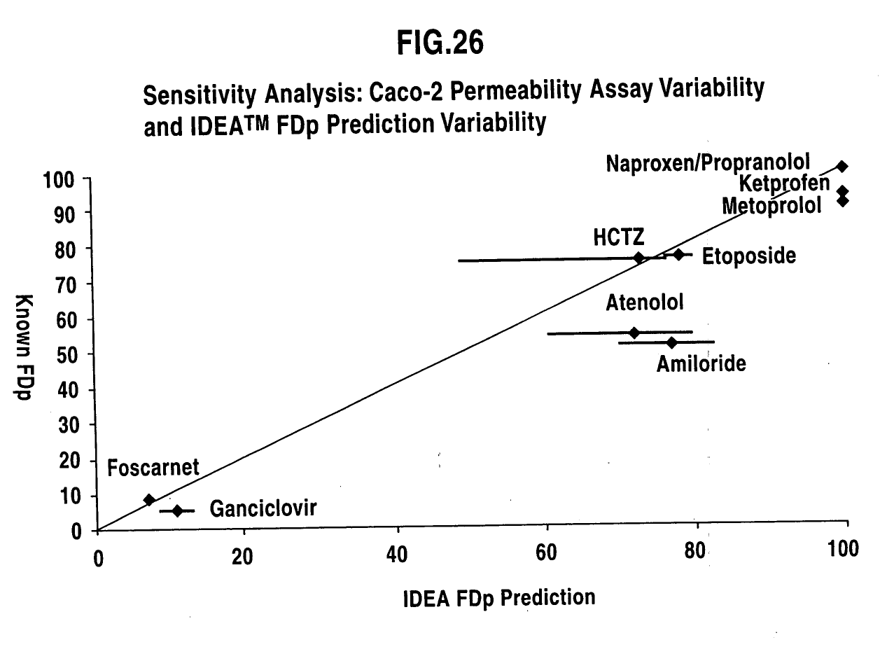

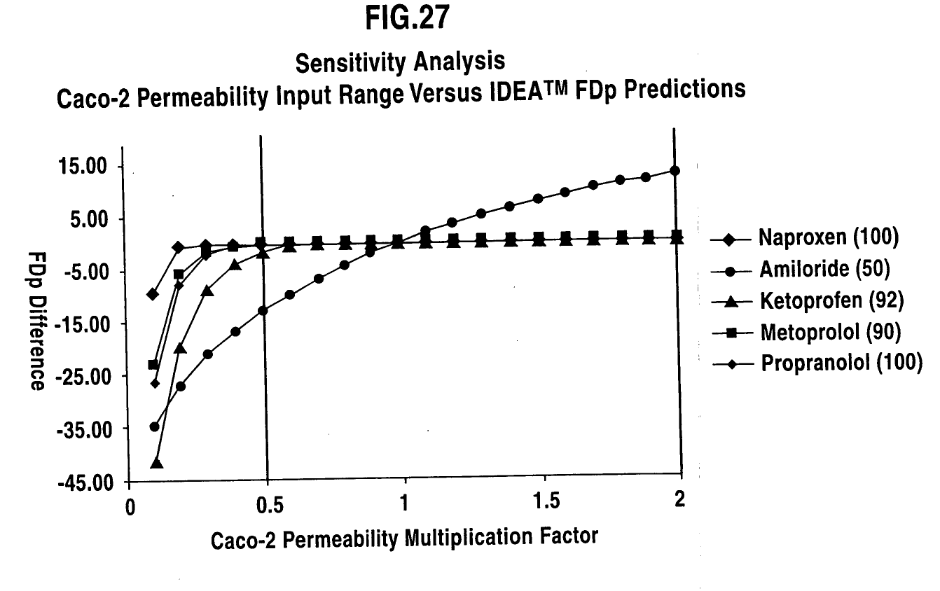

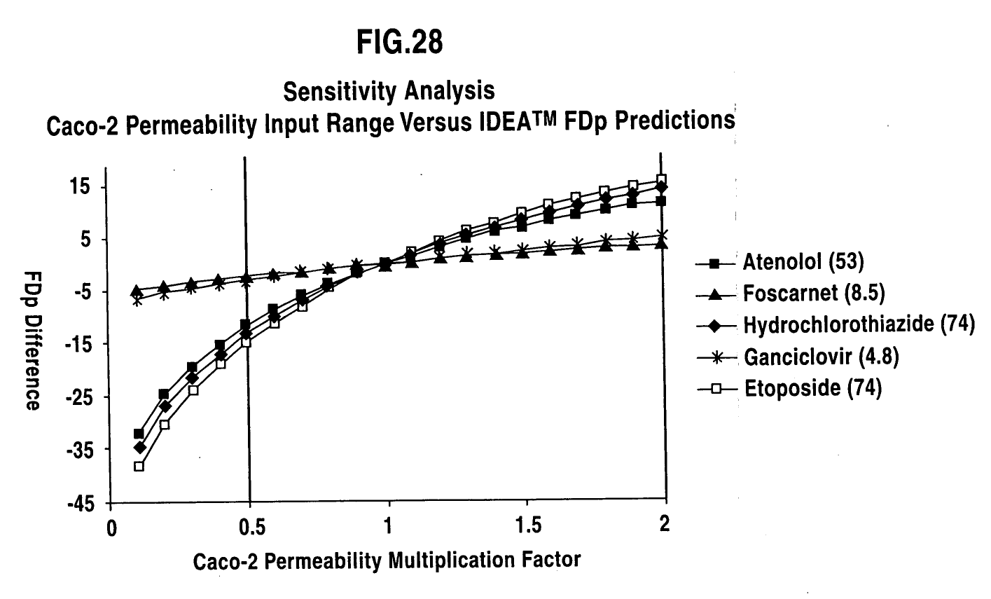

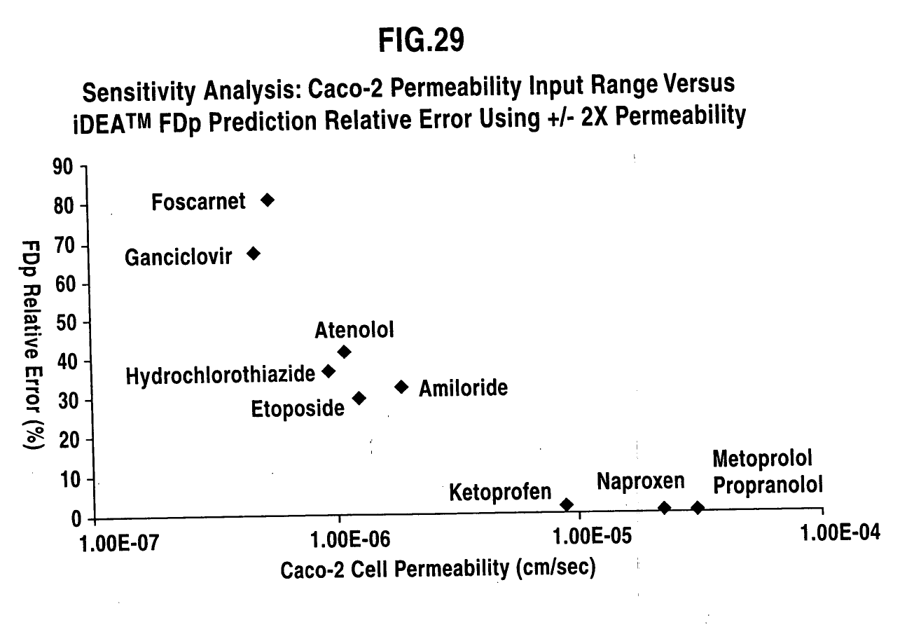

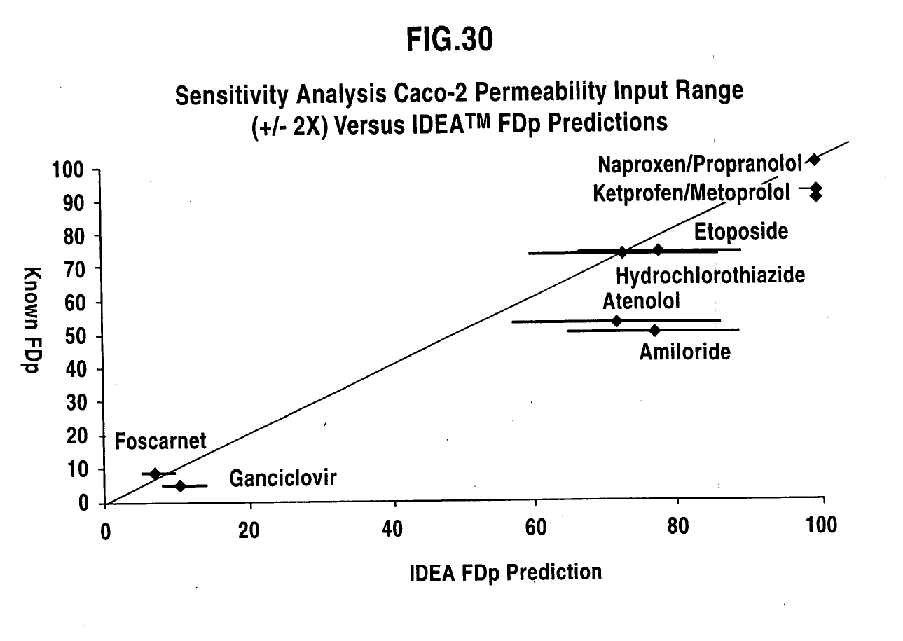

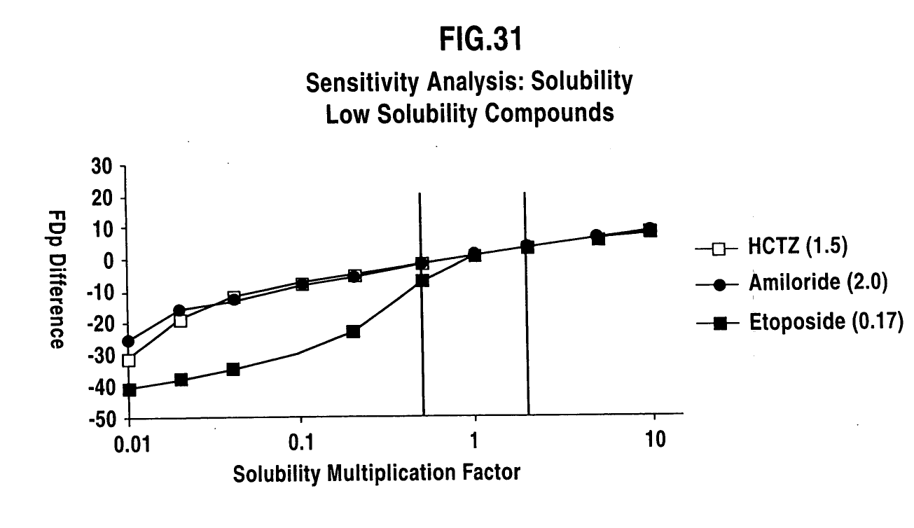

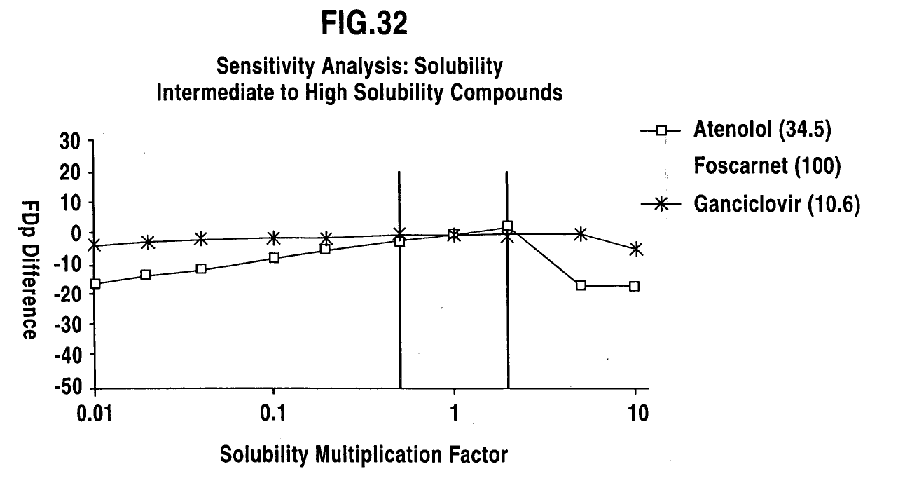

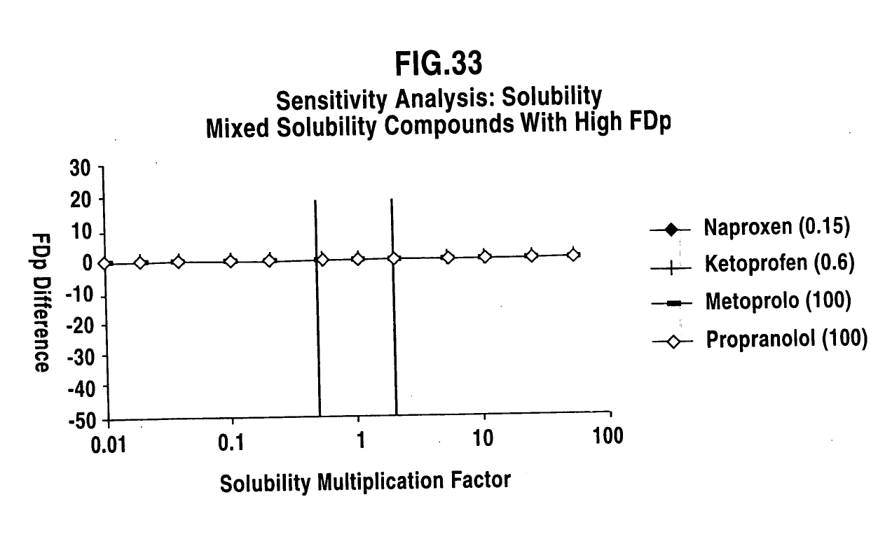

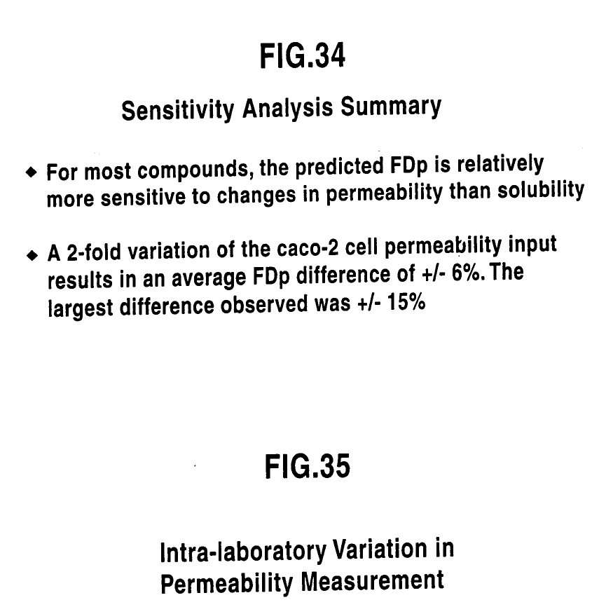

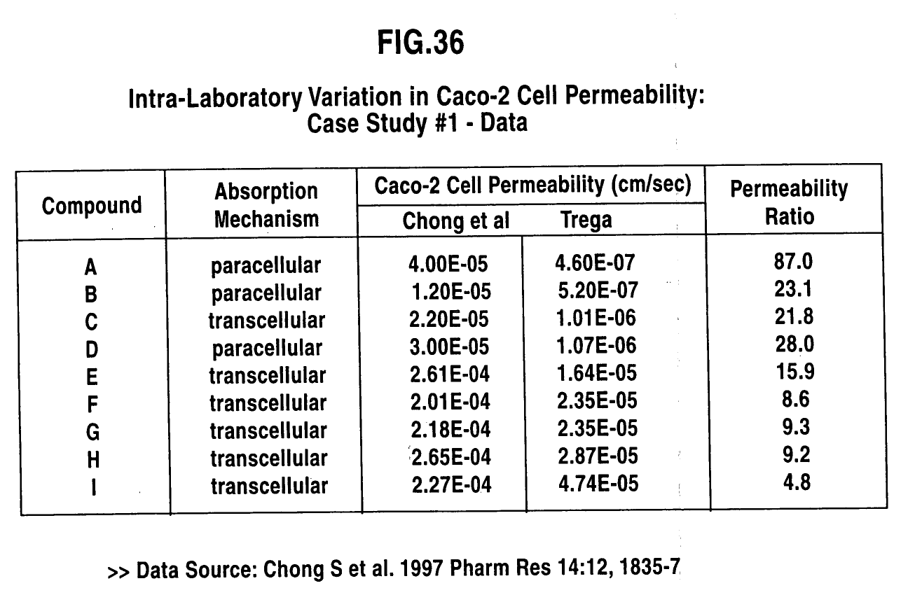

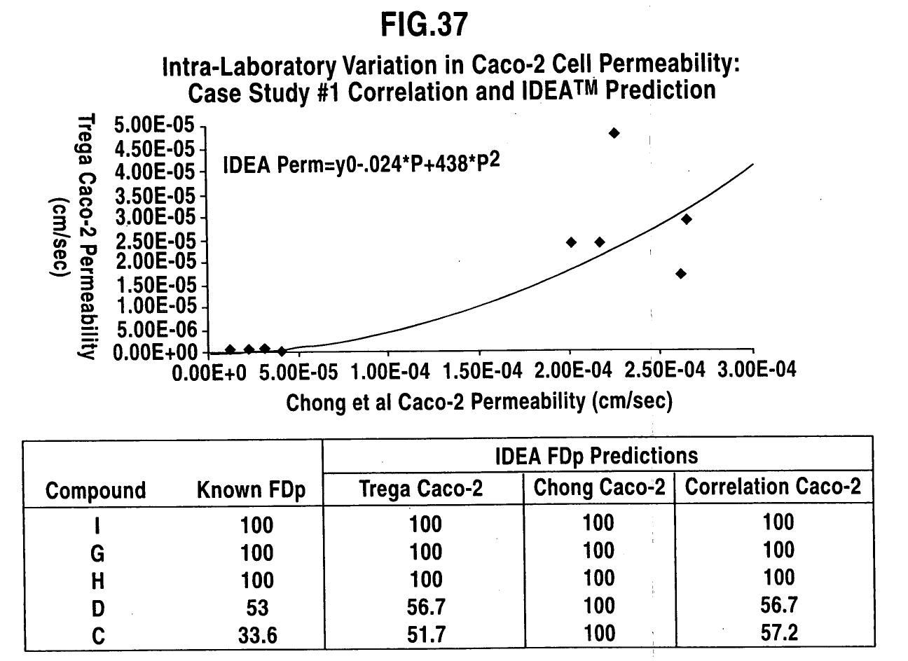

FIGS. 24-37 illustrate the sensitivity of an absorption model based on the model disclosed in the U.S. and PCT applications referenced above to changes and/or differences in the measured CACO-2 permeability, which result from differences in COCO-2 permeability data obtained from different sources. The CACO-2 permeability's were mapped into GI tract permeability's using the CACO-2 regional intestinal permeability model.

The CACO-2 regional intestinal permeability model may be improved by incorporating the molecular descriptors into the model. Thus, the second embodiment of the present invention would employ molecular descriptors, multiple in vitro assays such as CACO-2 and solubility for a compound to model, predict and/or estimate the compound's permeability in the various regions of a mammalian GI tract.

There are roughly four major properties involved in human pharmacokinetics: Absorption, Distribution, Metabolism, and Elimination (ADME). For example, when a drug is taken into the body orally, the first thing that has to happen is it has to get absorbed into the body in GI tract. From there, the drug travels to the liver via the portal vein where it is either metabolized or not. After the drug passes through the liver it is distributed throughout the body. Once the drug is distributed throughout the body, it is transported to the kidney to get eliminated. The effectiveness of a drug (a chemical compound) is directly related to the way a body will absorb, distribute, metabolize and eliminate the compound. In addition to the ADME properties of a compound, the toxicological effects of the compound should also be considered. The present invention is directed to systems and methods for predicting various characteristics (ADME/Tox characteristics) related to the way a body will absorb, distribute, metabolize, eliminate, and respond to potential toxic effects of a compound based on the compound's chemical structure and/or associated experimental data.

The molecular structure of a proposed compound may be input as a 2-dimensional (2D) connection table, which is essentially a two-dimensional graph of how the atoms of a compound are arranged (the structures may actually be 3-dimensional (3D), but may be represented as 2D via well known methods). Alternatively, the structure may be input as a 3D structure. Either 2D or 3D structural representations are desirable inputs for models using structure to predict ADME/Tox characteristics.

There are really three fundamental properties of the molecule that decide whether or not it's a drug: the first is whether or not it actually interacts with a particular molecular target in the body (in most cases, some kind of protein); the second is whether or not the body can absorb, metabolize, distribute and eliminate the compound adequately, and third, whether or not the compound elicits a toxic response.

The present invention provides systems and methods for predicting the ADME/Tox properties (e.g., Caco-2 effective permeability or Caco-2 Peff), of a proposed compound through statistical analysis of compound data. By using the present invention, it is therefore possible to significantly reduce the need for expensive and time consuming testing, such as animal testing, because the ADME/Tox characteristics of an untested compound is predicted with a high level of accuracy.

The first section of the present invention employs mathematical analyses of a diverse compilation of training data (chemical compound data including conventional experimental results, chemical descriptor analysis, etc.) to determine what data relates to the ADME/Tox property to be predicted. Once the type or types of data that are applicable to the ADME/Tox property (descriptors) are determined, mathematical analyses of the selected training data to obtain the selected ADME/Tox characteristic for each training data compound are performed in order to create a model. The model can then be used to predict a proposed compound's ADME/Tox property by inputting the same type of data for the proposed compound into the model. Running the model with the proposed compound's descriptors produces the predicted ADME/Tox characteristic.

Models are only as good as the input assay and test data, and therefore, a key to producing highly accurate predictions is the use of well-defined standard operating procedures for generating data as well as insuring that the data has a good distribution. Therefore, the present invention provides a method for collecting and compiling a diverse training data set to be used to mathematically predict the ADME/Tox characteristics of a proposed chemical compound.

The input data is collected and/or calculated for a variety of chemical compounds preferably representing currently prescribed drugs as well as failed drugs and potential new drugs (this is a continual process, since as more data is collected, the resulting models will have improved performance). Assay data may be collected from well established sources or derived by conventional means. For instance, in vitro assays characterizing permeability and transport mechanisms may include in vitro cell-based diffusion experiments and immobilized membrane assays, as well as in situ perfusion assays, intestinal ring assays, incubation assays in rodents, rabbits, dogs, non-human primates and the like, assays of brush border membrane vesicles, and averted intestinal sacs or tissue section assays. In vivo assay data typically are conducted in animal models such as mouse, rat, rabbit, hamster, dog, and monkey to characterize bioavailability of a compound of interest, including distribution, metabolism, elimination and toxicity. For high-throughput screening, cell culture-based in vitro assays or biochemical assays from isolated cell components or recombinantly expressed components are preferred. For high-resolution screening and validation, tissue-based in vitro and/or mammal-based in vivo data are preferred.

Cell culture models are preferred for high-throughput screening, as they allow experiments to be conducted with relatively small amounts of a test sample while maximizing surface area and can be utilized to perform large numbers of experiments on multiple samples simultaneously. Cell models or biochemical assays also require fewer experiments since there is no animal to animal variability. An array of different cell lines also can be used to systematically collect complementary input data related to a series of transport barriers (passive paracellular, active paracellular, carrier-mediated influx, carrier-mediated efflux) and metabolic barriers (protease, esterase, cytochrome P450, conjugation enzymes).

Cells and tissue preparations employed in the assays can be obtained from repositories, or from any eukaryote, such as rabbit, mouse, rat, dog, cat, monkey, bovine, ovine, porcine, equine, humans and the like. A tissue sample can be derived from any region of the body, taking into consideration ethical issues. The tissue sample can then be adapted or attached to various support devices depending on the intended assay. Alternatively, cells can be cultivated from tissue. This generally involves obtaining a biopsy sample from a target tissue followed by culturing of cells from the biopsy. Cells and tissue also may be derived from sources that have been genetically manipulated, such as by recombinant DNA techniques, that express a desired protein or combination of proteins relevant to a given screening assay. Artificially engineered tissues also can be employed, such as those made using artificial scaffolds/matrices and tissue growth regulators to direct three-dimensional growth and development of cells used to inoculate the scaffolds/matrices. It will be understood that ideally any known test results could be added to a test data set in order to adjust the model or to provide a new property to solve towards.

The drugs (compounds) selected should be as diverse in character as possible. Therefore, the compounds may be analyzed and defined in chemical space. Chemical space can be represented as an N-base coordinate system in which to plot compounds and may be used to show the diversity of a sample of compounds. The axes of N-base coordinate system may be selected from all or some of the input data. Drugs may be eliminated from a particular training data set (the training data may be grouped to solve for a particular ADME/Tox property) if it is determined that they bias the training data set.

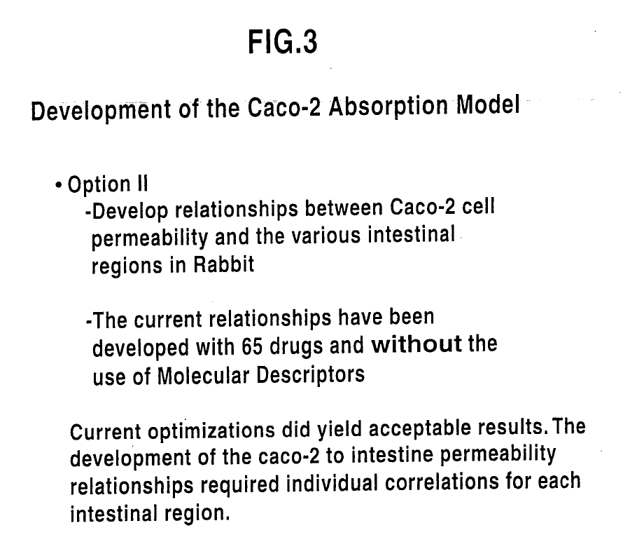

In the present invention, a collection of drugs have been plotted in a six-base chemical space (see FIG. 3). The axes of the six-base are physicochemical descriptors that were selected so that the best separation of known drugs is maintained. Data is also selected from combinatorial libraries of chemicals which are near neighbors for each of the drugs creating an extended data set. The compounds are ideally each tested for various ADME/Tox characteristics or properties to be predicted, however it is not necessary to test every compound for actual results.

There are many considerations for the experimental data. Each data set of experimental data is analyzed to decide how it is going to be used in model building. For example, is it appropriate to use a certain data set to predict absolute values of compounds or is there too much error in the data set? If there is not enough data in a data set to cover a particular range (either coverage in the data space, representation in the data space, or certainty in the data space) it is possible to put the data into bins, such as 0 to 20, 21 to 40, 41 to 60, 61 to 80, 81 to 100. Alternatively, the data may require scaling correction to account for systematic variations in the data. One having ordinary skill in the art will readily understand the grouping of experimental data, scaling and systematic variations used to adjust a data set.

Next, a tool is used to calculate additional data by analyzing each compound and describing the compound with chemical descriptors. Chemical descriptors are well known in the art of modeling compounds, and may be determined by analyzing a 2D or 3D structure of a compound.

Finally, all the training data (input and target data) collected or created is compiled and preferably maintained in a relational database or other known means for making the data easily accessible and available to be manipulated and analyzed in accordance with the present invention.

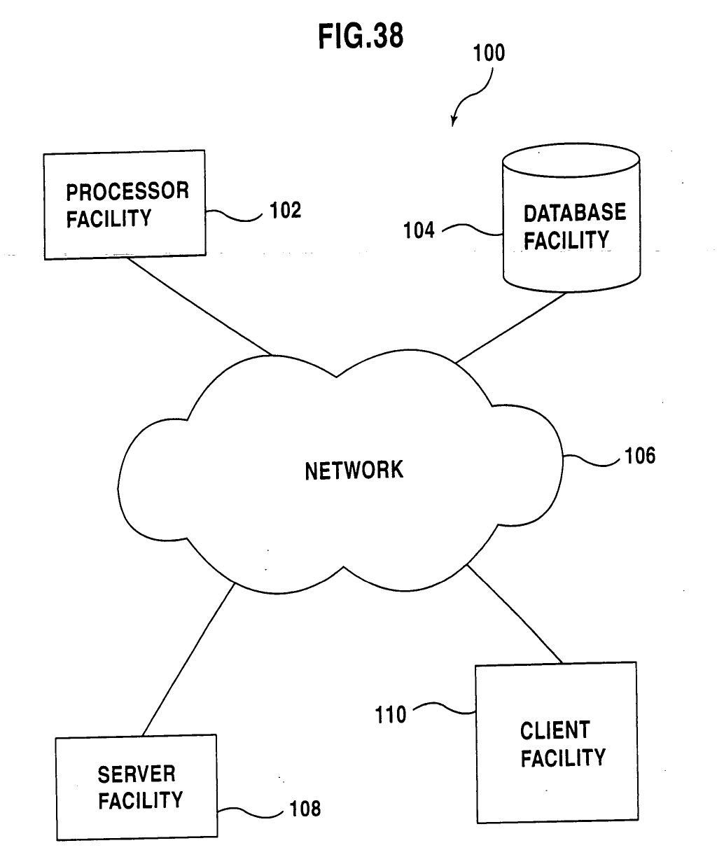

The present invention is now described with reference to FIG. 38. In particular, system 100 includes a processor facility 102 and a data facility 104 coupled to a network 106. The processor facility 102 may be a conventional computer, such as a PC, configured to access database facility 104 and to execute analytical software in accordance with the present invention. Database facility 104 may be a conventional database server running a database engine, such as SQLSERVER® or ORACLE 8i®) and is configured to maintain and to serve data, such as the test data described above. The data may be stored and maintained by any means such as in a relational dataspace or an objected oriented dataspace.

The present invention includes analytical tools which may be executed on processor facility 102. The analytical tools may be in the form of software that is loaded locally on processor facility 102 or may be served via a server 108 (e.g., an HTML form, JAVA program, etc. served on a web server), which optionally may be included. Accordingly, a client facility 110 may be connected to the network 106, which may include parts of the Internet and World Wide Web (WWW), or local area networks (LANS). The client facility 110 could be a web browser or other terminal configured to access and run the analytical tools remotely or to download the analytical tools (e.g., via HTML, IIOP, etc.) via network 106 and run them locally.

The configuration of system 100 is merely exemplary and is not meant to limit the present invention. It will be appreciated that the present invention may take many forms and configurations. For example, the present invention may be implemented via a software solution including a database and forms configured to run on a stand-alone PC, or may alternatively be a combination of software and firmware, and may be implemented in a client-server, stand-alone or web configuration.

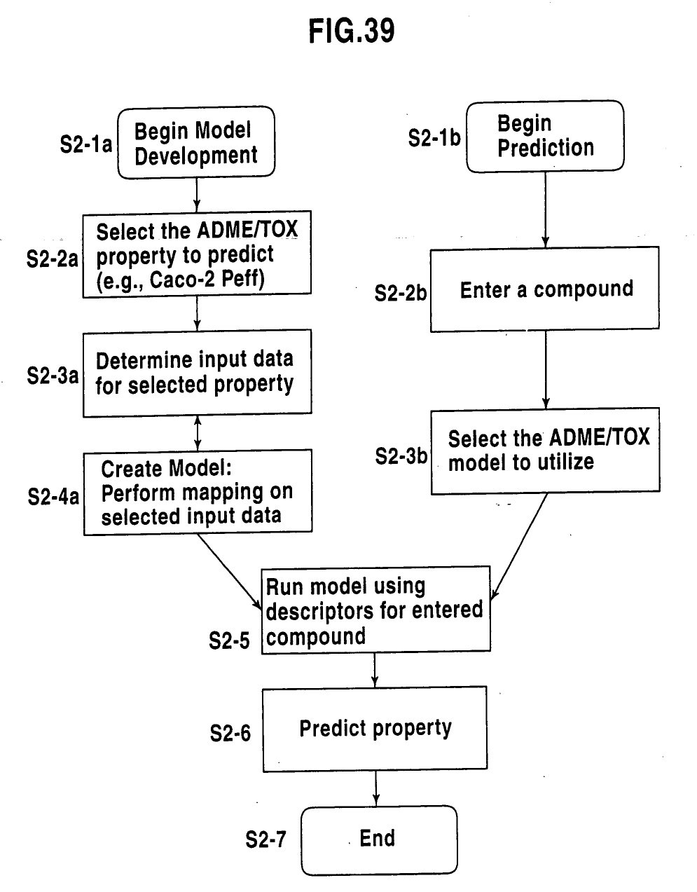

The operational aspects of the present invention are now described with reference to the flow chart in FIG. 39. The flow chart represents two independent starting pathways which meet at step S2-5, a model development pathway, and a model execution or prediction pathway, these two initial pathways will be described independently.

Model Development Pathway (S2-1a->S2-5)

The model development pathway begins in step S2-1a and immediately proceeds to step S2-2a. At step S2-2a, the ADME/Tox property to be predicted is selected. For example, it may be desired to predict the Caco-2 Peff of the compound, or the FDP (fraction of the dose administered that is absorbed at the portal vein). The system might allow for the selection to be from a table, radio group, pop-list, or by any known means. Also at step S2-2a, a set of training compounds appropriate for developing the selected ADME/Tox property model is entered into the system. Many compound descriptors may be entered or calculated, such as molecular weight, structure, specific gravity, etc.

Next, at step S2-3a, a group of meaningful input data is selected based on the property to be predicted or a related performance metric using feature selection methods. For example, a genetic algorithm coupled with a regression/classification method, such as a neural network, may be used to build many models predicting the Caco-2 Peff of a compound. Features are then selected from the resulting models with the objective of choosing the smallest number of dimensions that effectively describe the model space. One should keep in mind when performing the analyses to select a number of descriptors which avoids biased and non-predictive models (e.g., overtraining).

Once the descriptors have been selected, a model is created at step S2-4a by using regression/classification methods to map the input data to the ADME/Tox property to be predicted. The modeling effort may involve Affine Regressions, Nearest Neighbor Methods, Discriminate Analysis, Support Vector Machines, Artificial neural networks, Data Compression techniques (targeted and non-targeted), Genetic Algorithms, and Boosting. In addition, a method for calculating a confidence metric is created by analyzing information related to the model such as the distributions and values of the input and target data and the methods involved in building the model.

It should be noted that instead of predicting continuous values for a specific ADME/Tox property, the present invention may be used to classify a particular compound (e.g., can it be absorbed, is it toxic, etc.). A compound is classified by the same method predicting a specific ADME/Tox property, except that the analyses performed may vary slightly, and the classifications are performed to solve for a “yes/no” or “high, medium, low” binning type solution (e.g., 1-bit).

The model resulting from step S2-4a is used in step S2-5 to predict new proposed compounds in the model execution pathway. Model Execution Pathway (S2-1b->S2-7)

Once the model has been created/developed, then the model may be used to predict the ADME/Tox property of the proposed compound. The model execution pathway begins at step S2-1b, and proceeds directly to S2-2b where at least one proposed compound may be entered.

Next, at step S2-3b, the property to be predicted is selected. For example, it may be desired to predict the Caco-2 Peff of the compound, or the FDP. The system might allow for the selection to be from a table, radio group, pop-list, or by any known means.

Next, at step S2-5, the descriptors for the proposed compound (identified in step S2-3a)) are input into the model created in step S2-4a. The model is run and a result (e.g., a Caco-2 Peff or FDP prediction) is produced in step S2-6. As described above, a measure of confidence in the result may also be produced.

Processing terminates at step S2-7.

It should be readily apparent to one having ordinary skill in the art that the preceding method may be implemented via numerous configurations. For example, the preceding method and analysis therein may be implemented via a C++ program coupled to a data warehouse, or alternatively may be implemented via a combination of program components and databases.

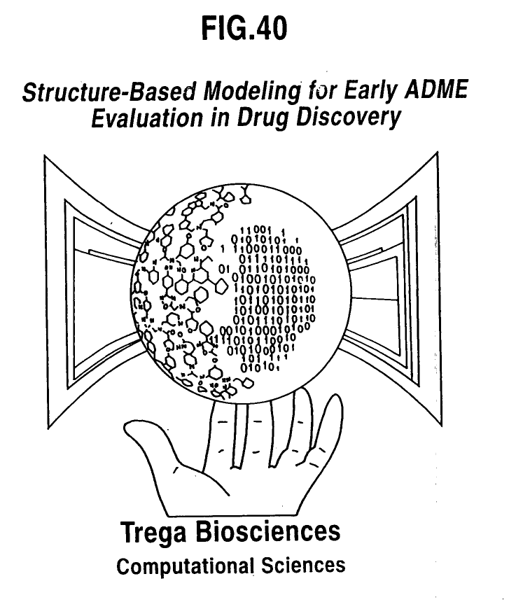

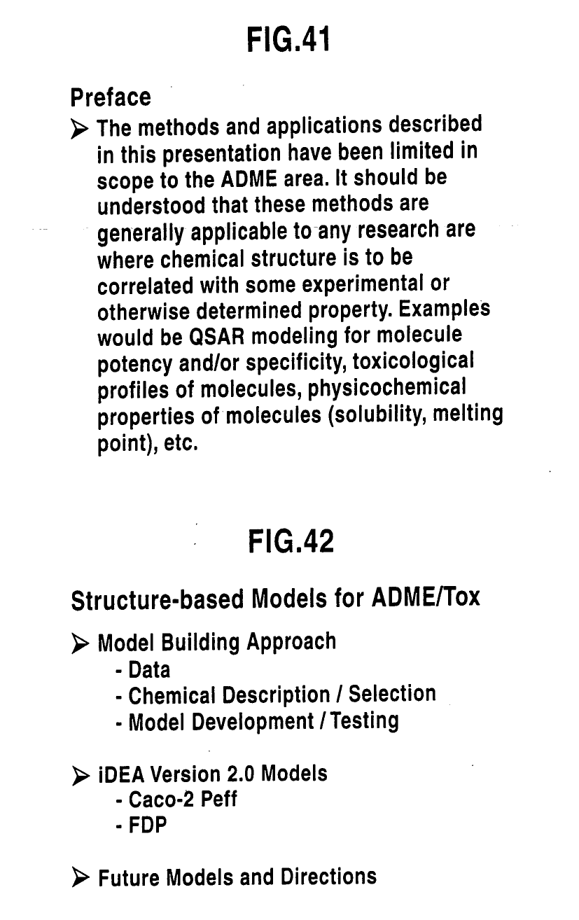

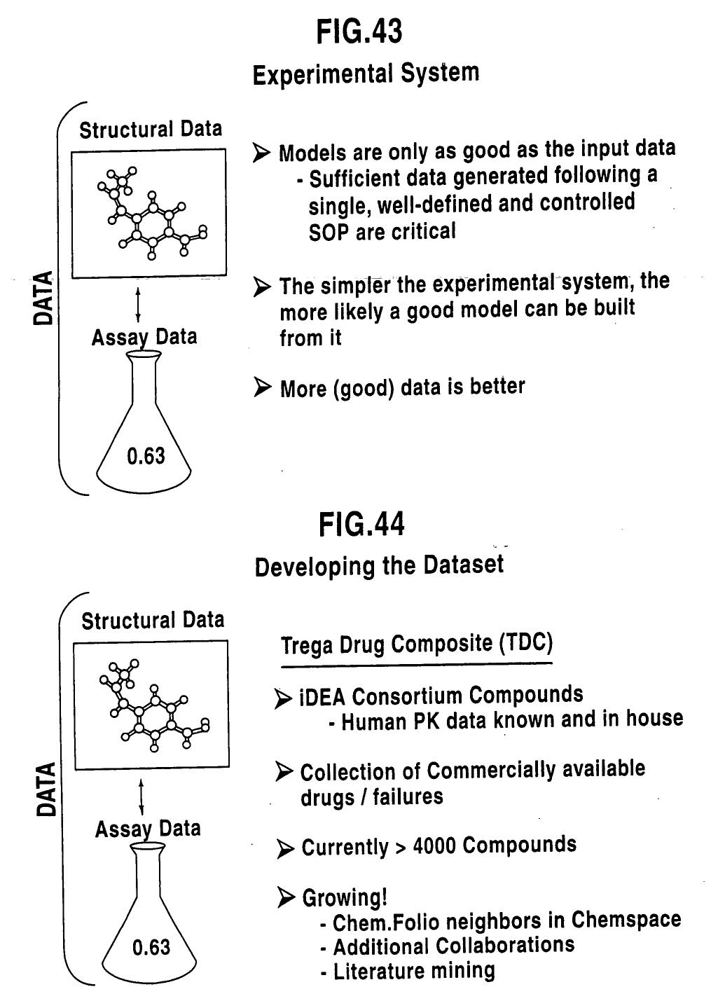

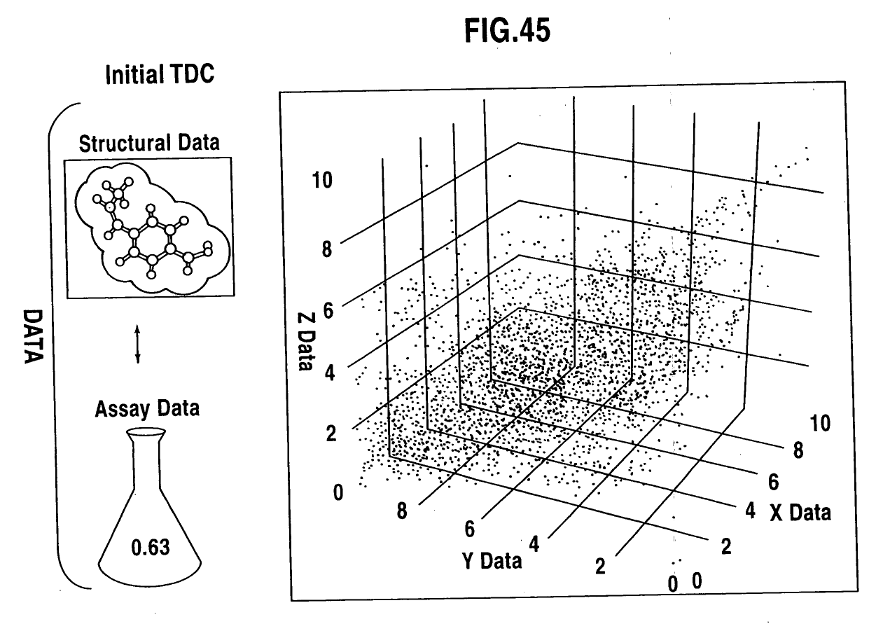

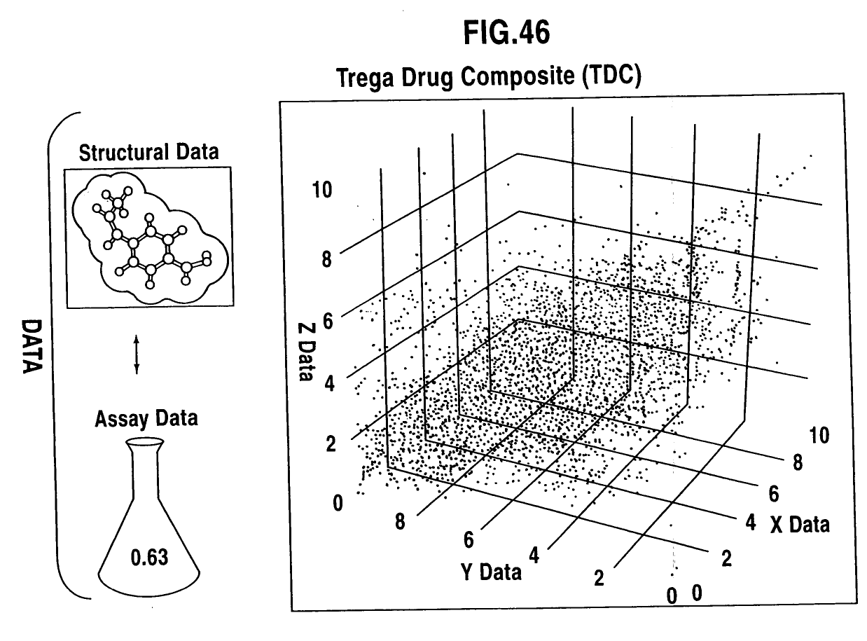

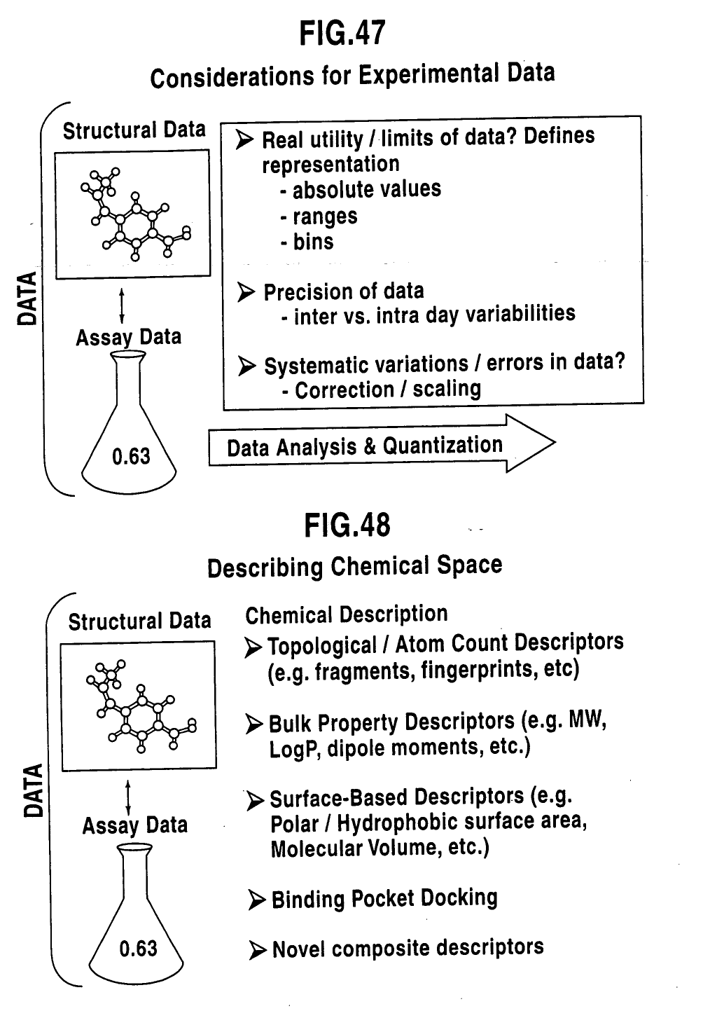

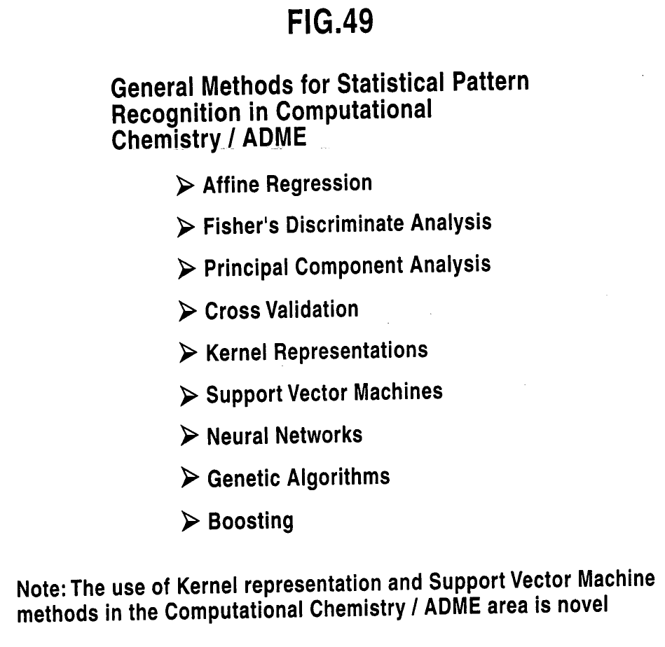

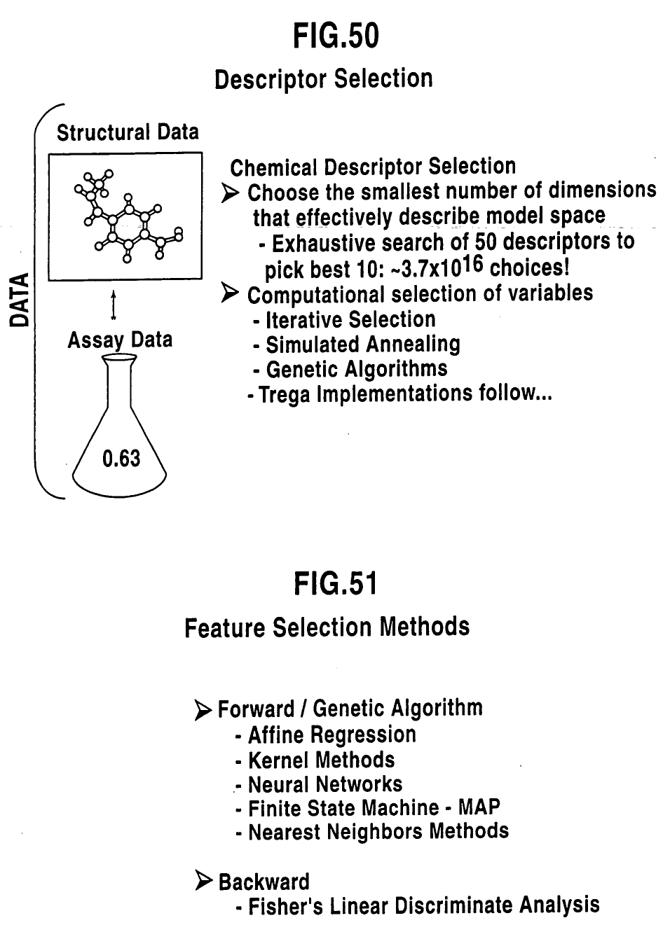

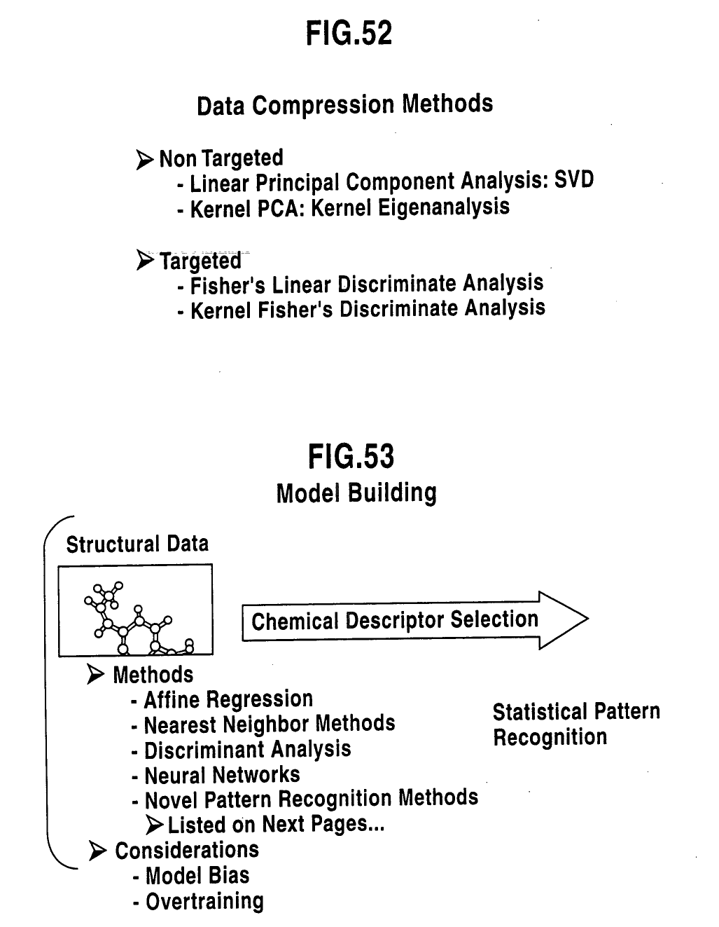

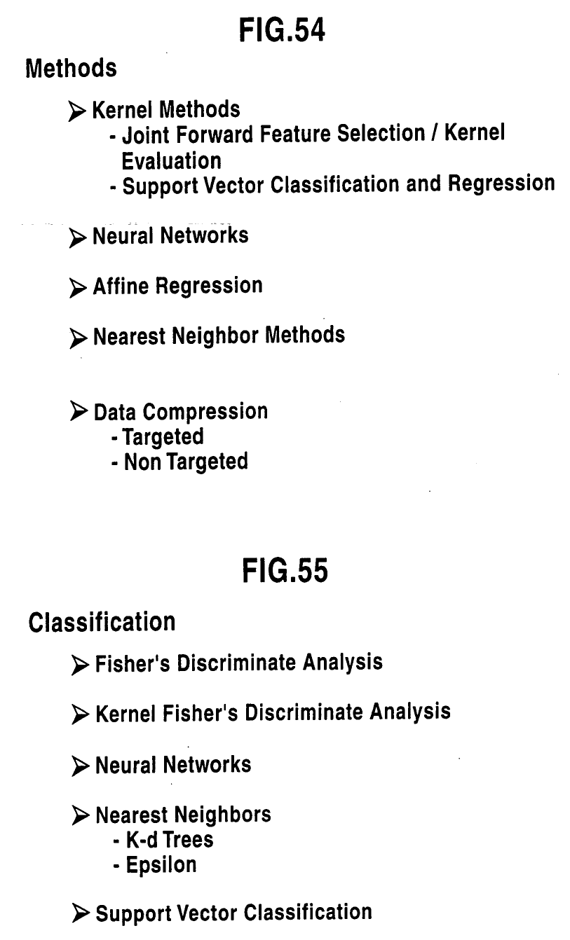

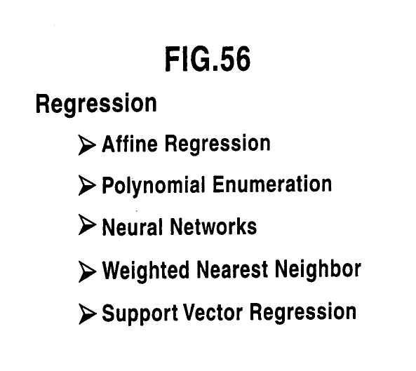

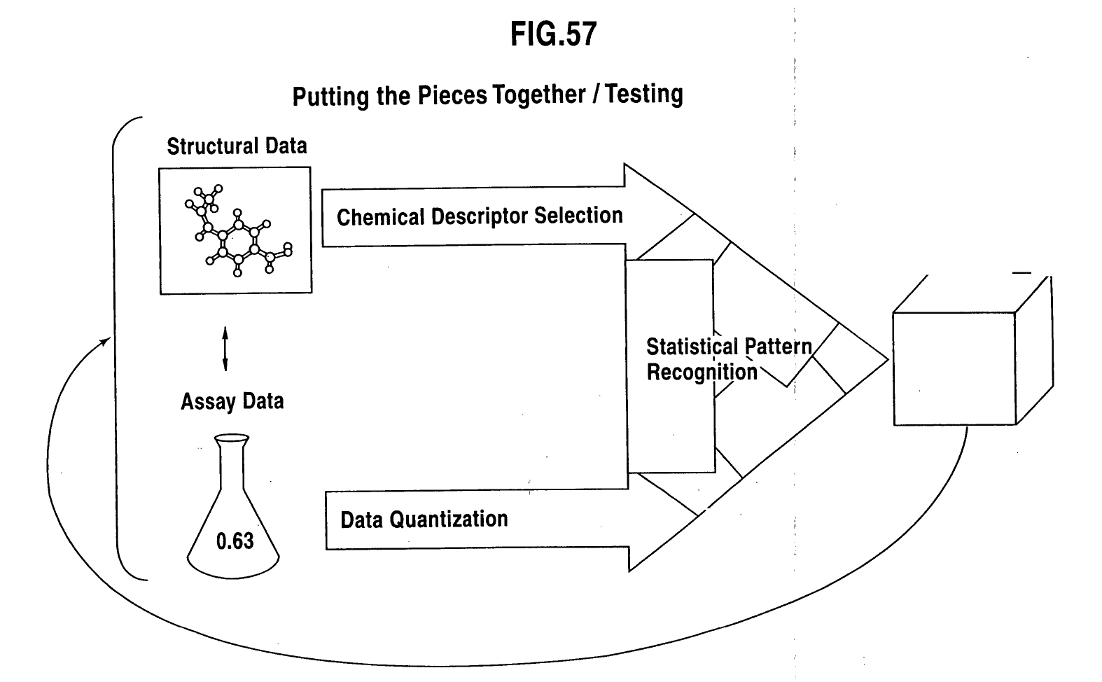

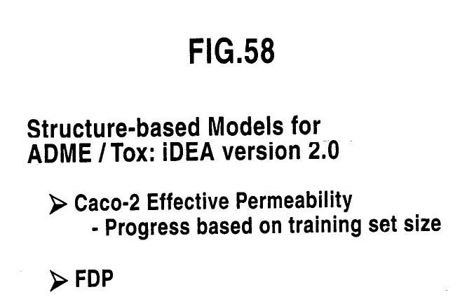

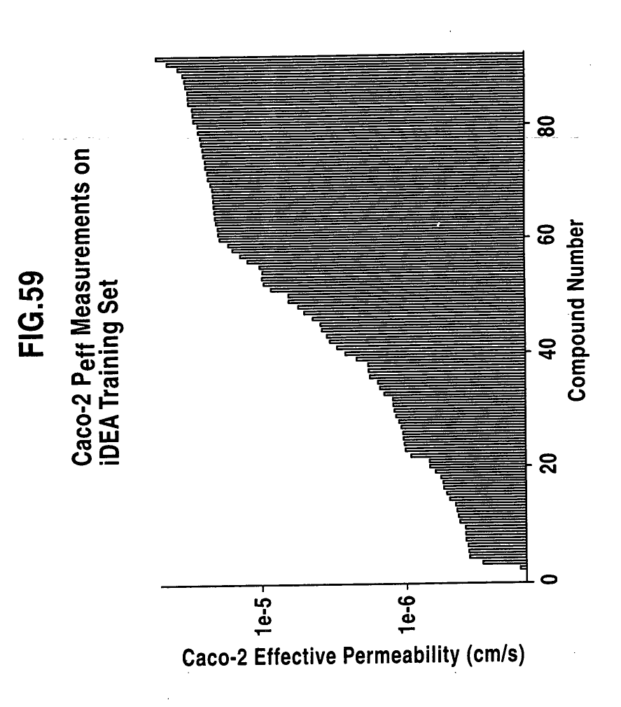

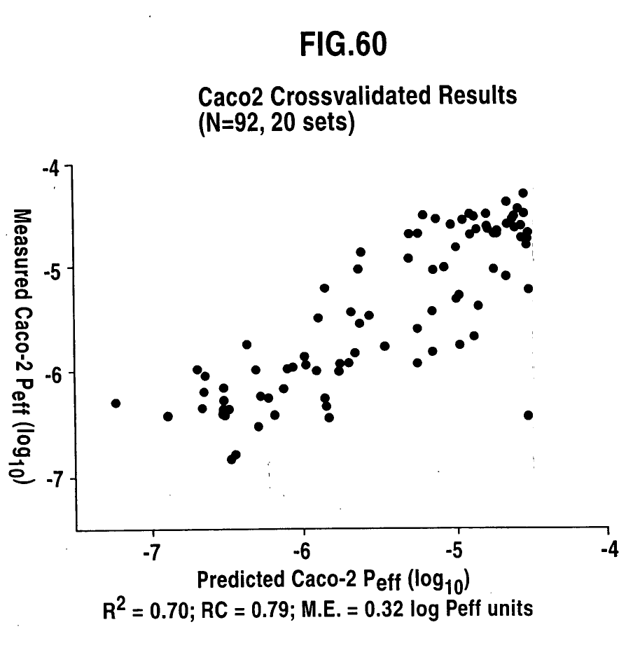

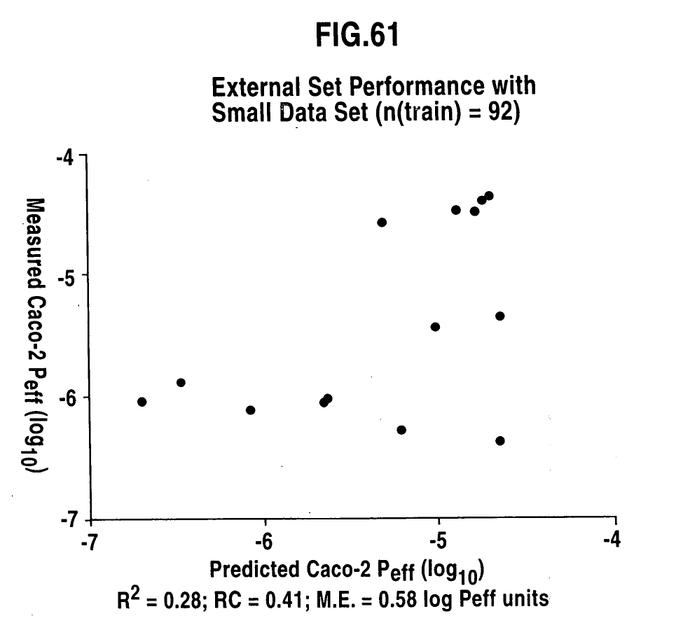

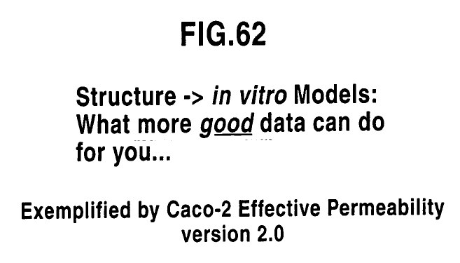

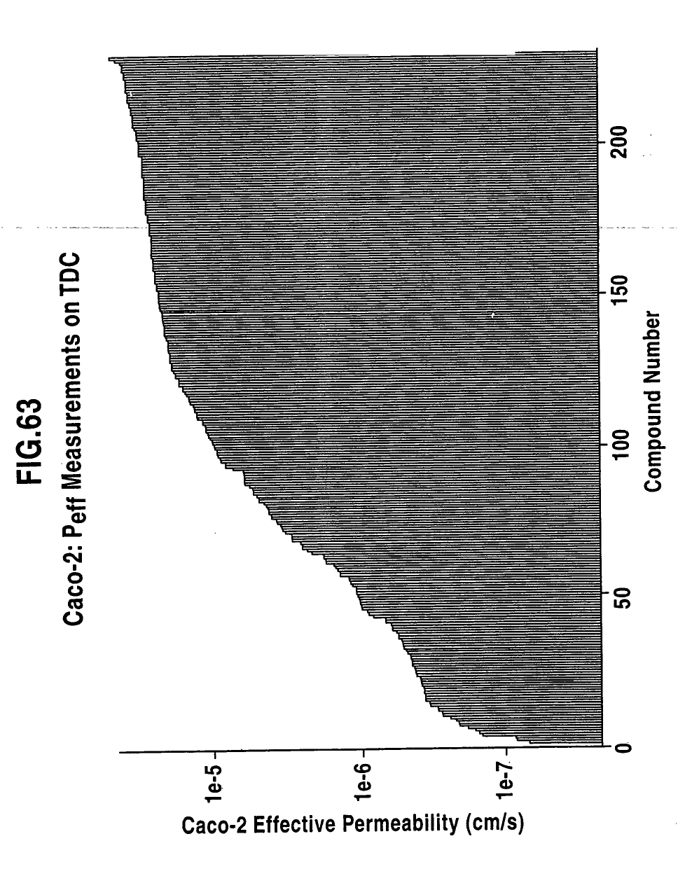

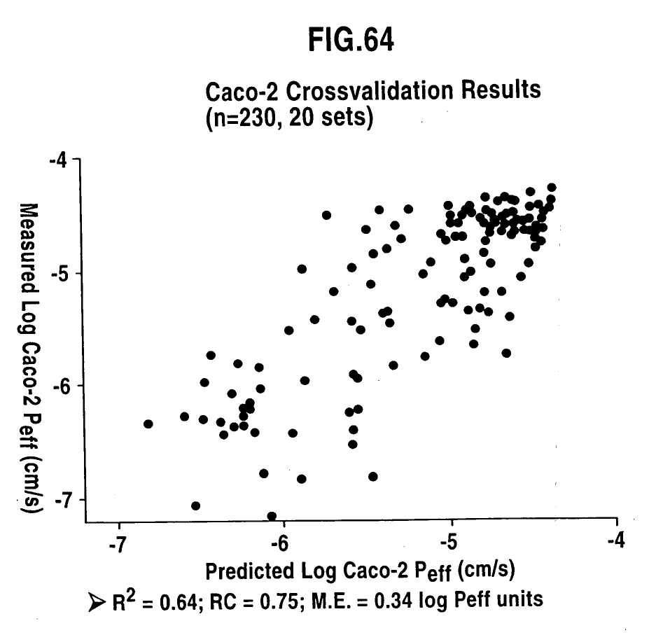

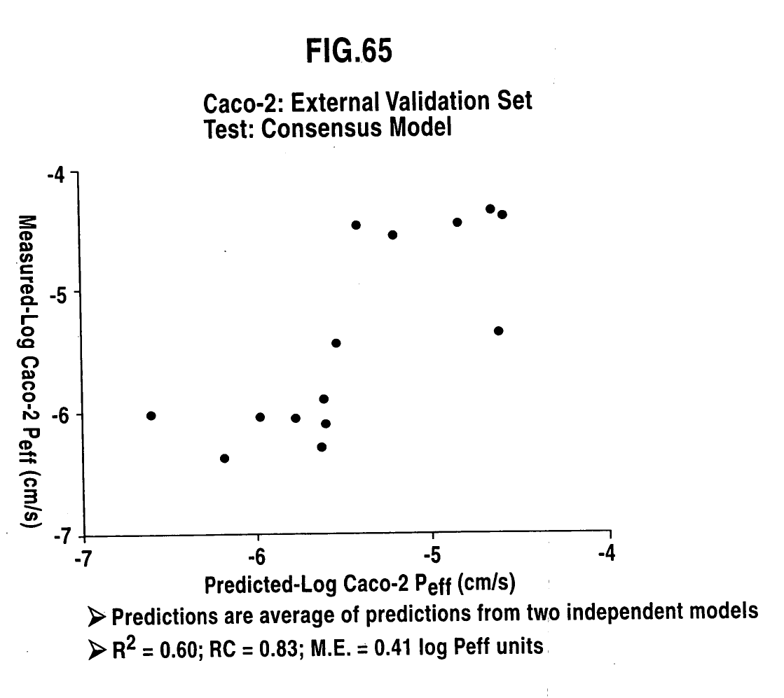

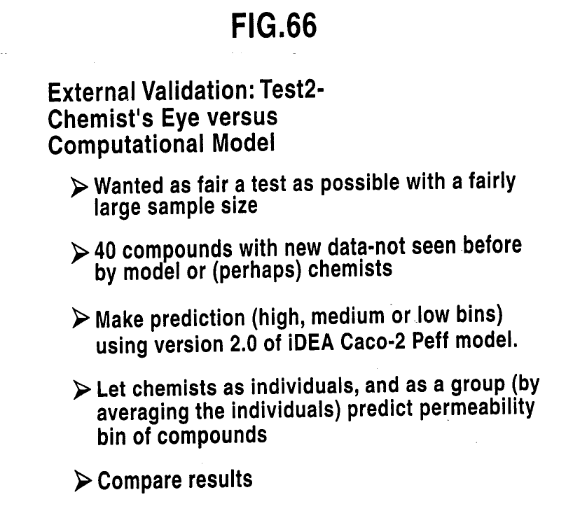

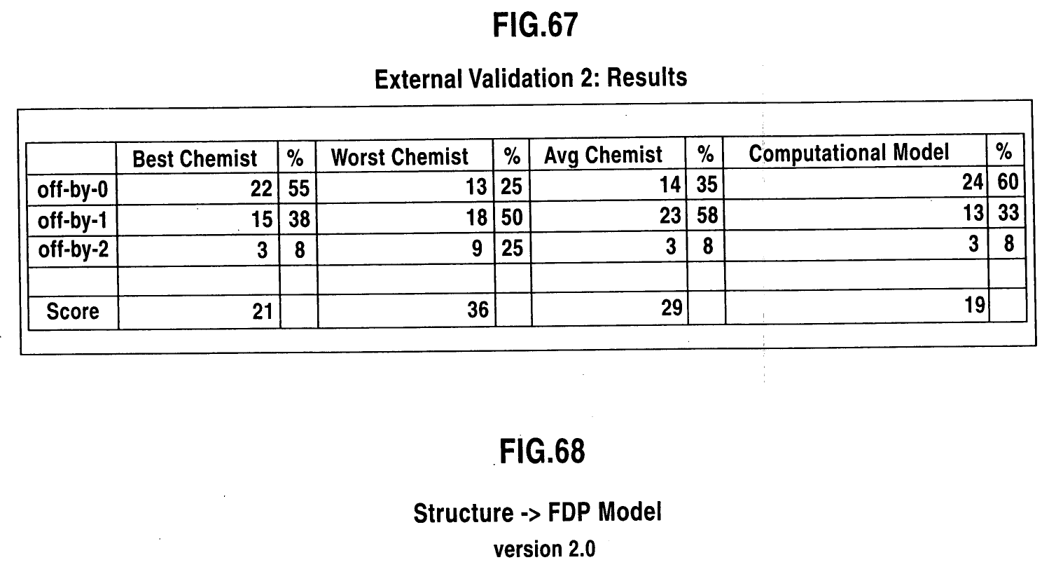

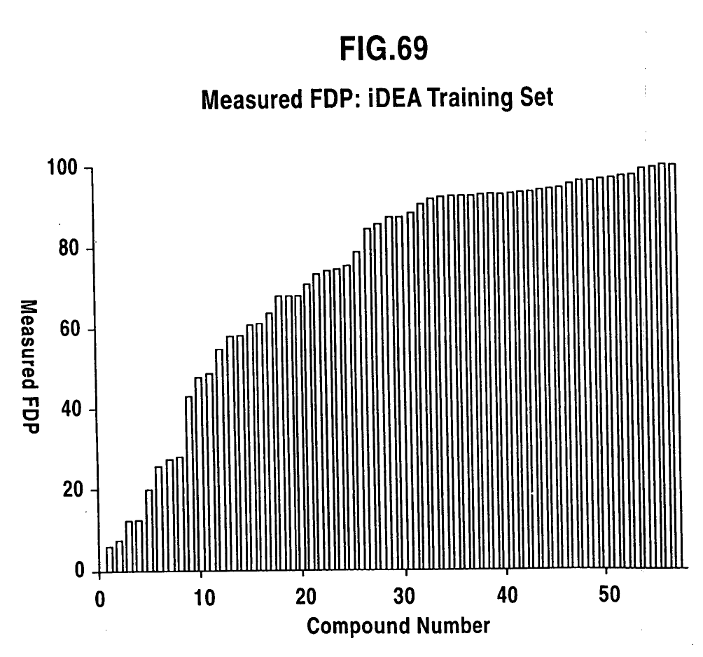

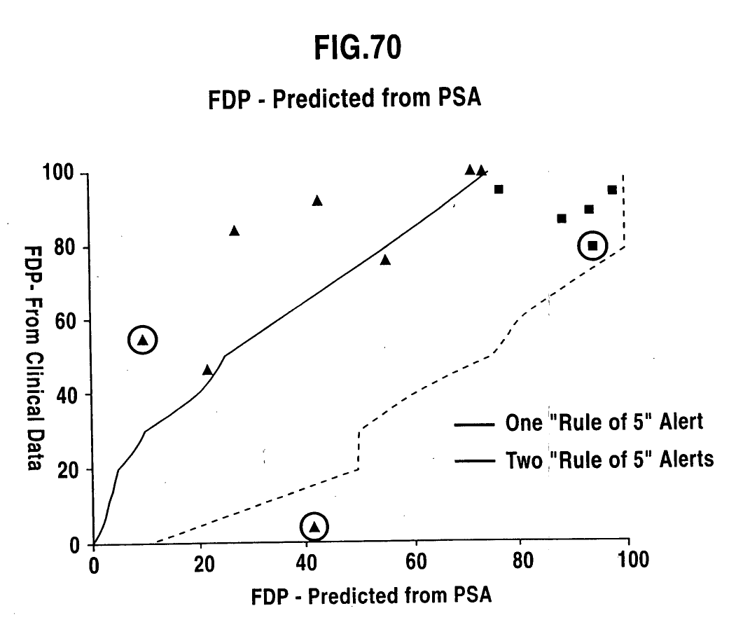

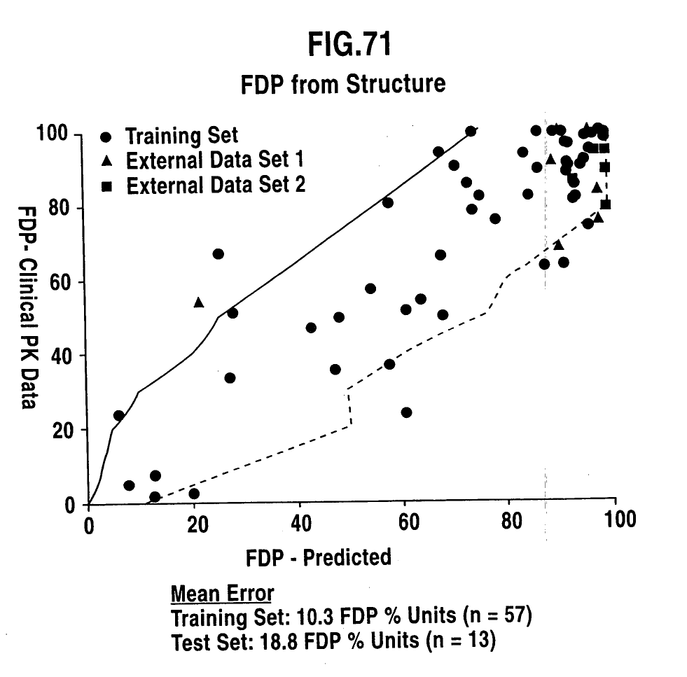

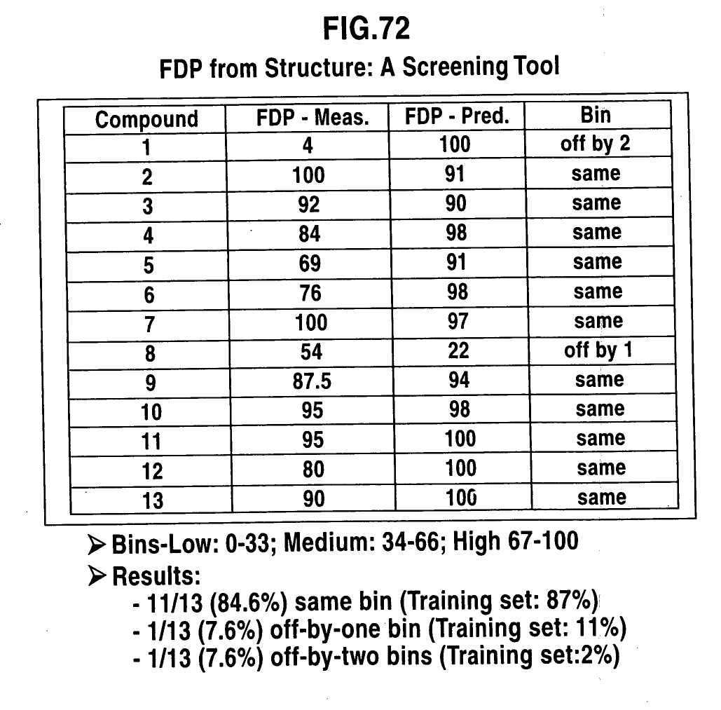

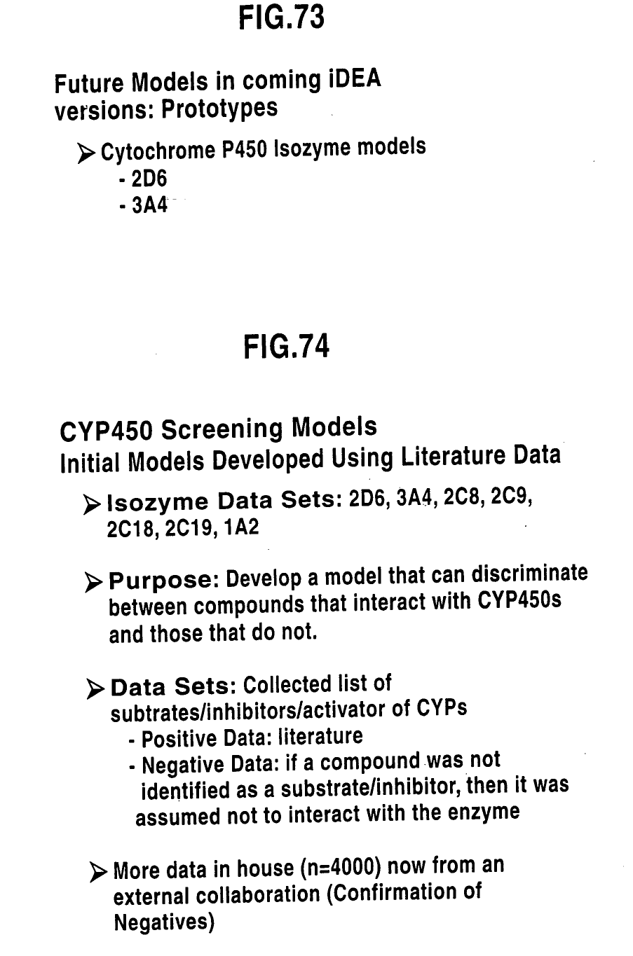

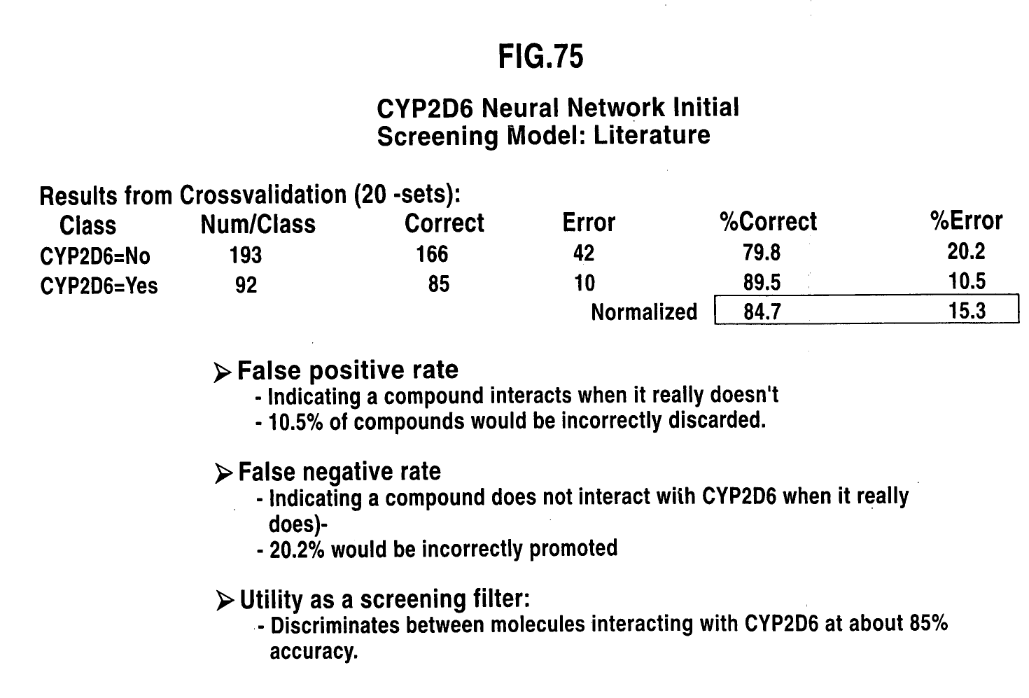

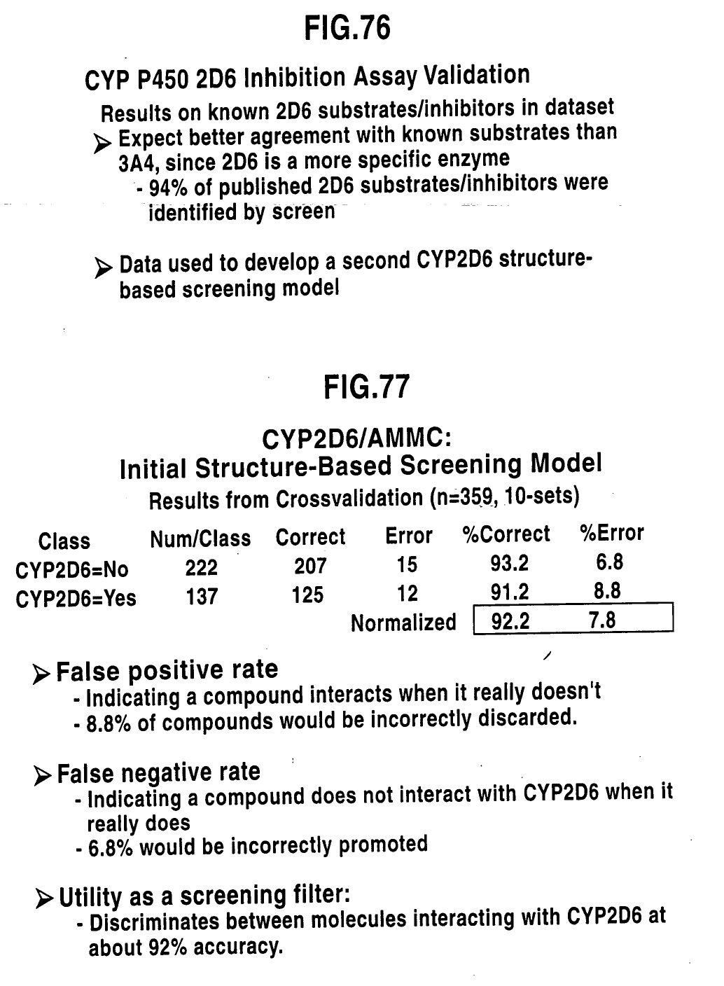

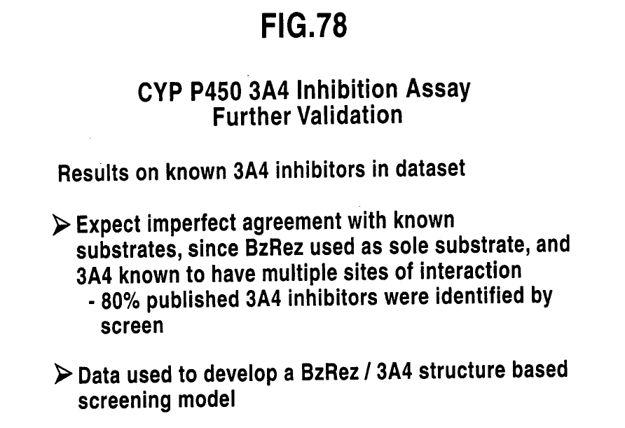

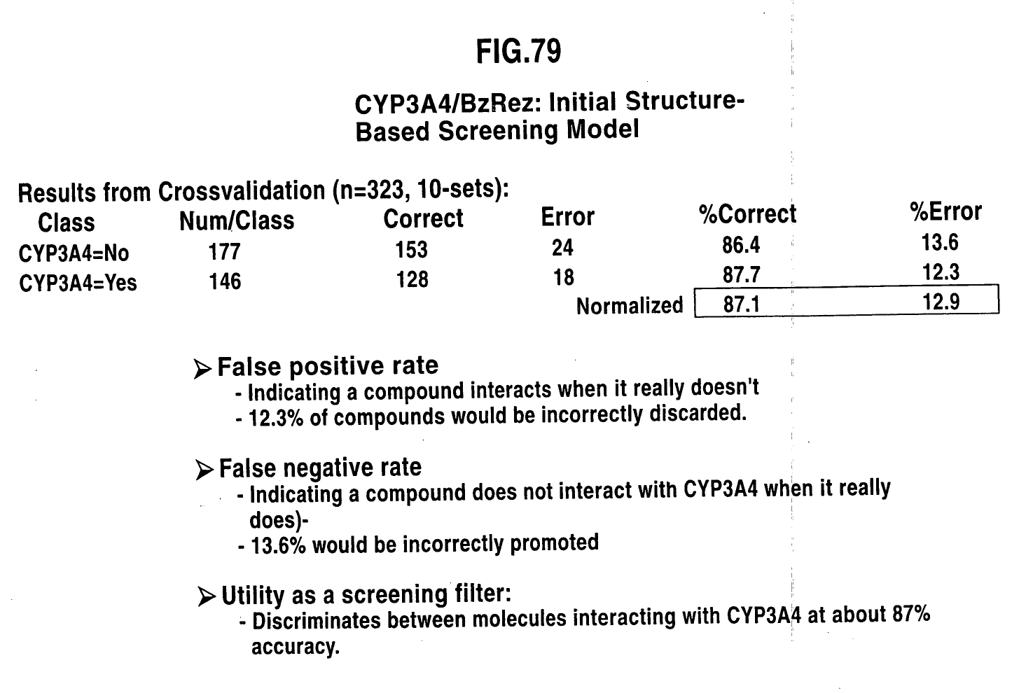

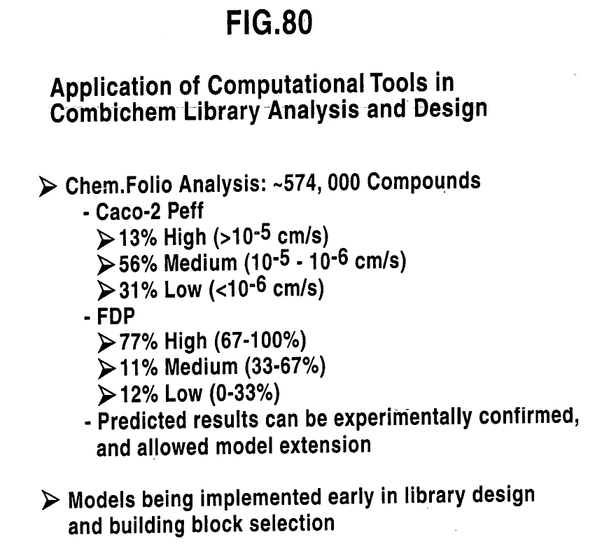

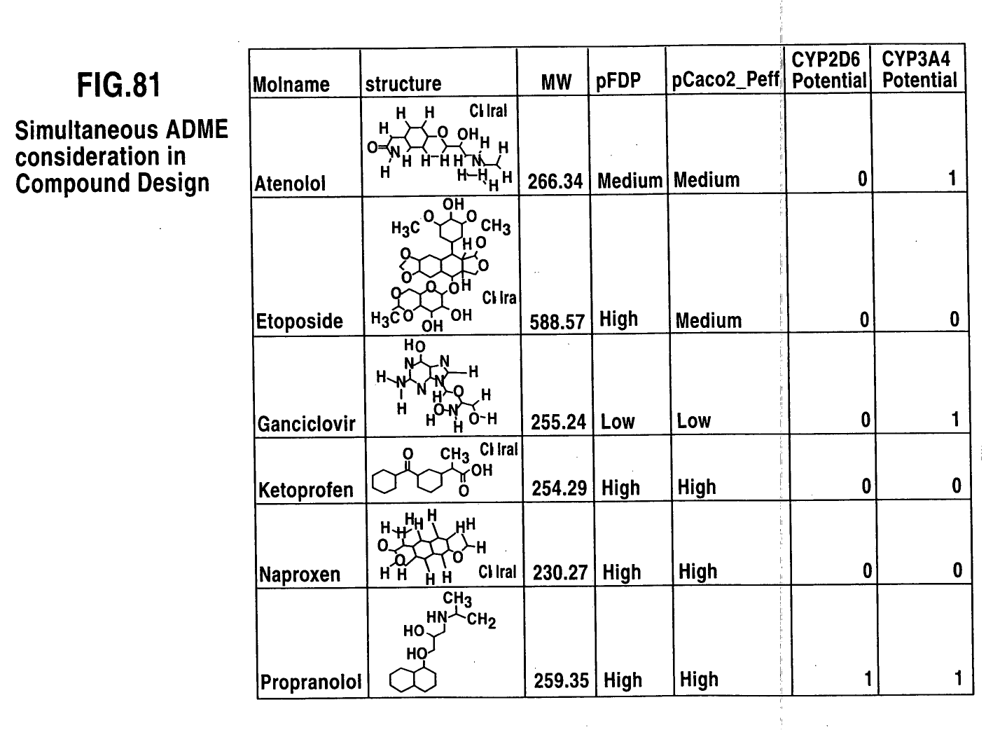

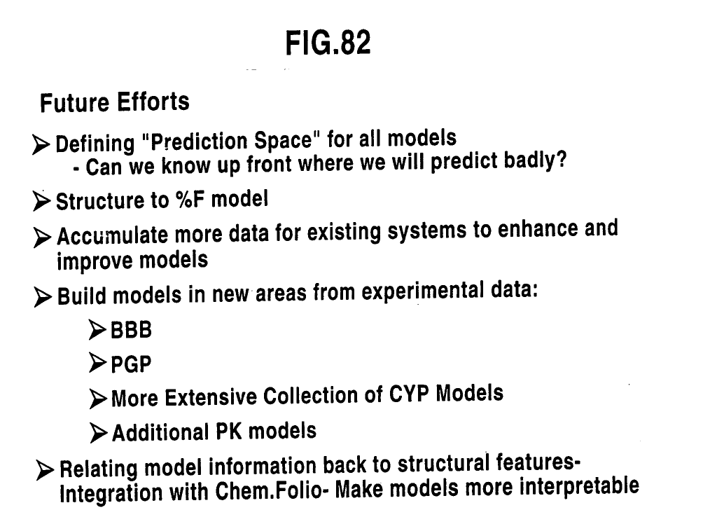

FIGS. 40-82 provide additional detail regarding model development.

The regional intestinal permeability model may be further improved by accounting for or providing correlations between the experimental conditions utilized to obtain the data employed to train and/or develop the model and the experimental conditions found and/or utilized by the model's user. The following paragraphs discuss the inter-laboratory differences in CACO-2 permeability data; the effect of pH, buffer, and CO₂ Incubation on CACO-2 permeability data; and Fasted State Simulated Intestinal Fluid on CACO-2 permeability data. It is expected that one of ordinary skill in the art could improve the basic regional intestinal permeability model described above to either account for the different conditions under which the CACO-2 permeability data input into the model is obtained or to correct (adjust) and/or correlate the CACO-2 permeability data input and the conditions under which the data was obtained. In the event that a correction and/or correlation to the CACO-2 permeability data input is made, a CACO-2 permeability value could be obtained by adjusting the CACO-2 permeability data input to account for know and/or expected experimental differences. Consequently, with consistent data input the output of the CACO-2 regional intestinal model should better reflect actual permeability. Therefore, the output of models employing the output of the CACO-2 regional intestinal model should also better reflect in vivo'conditions.

A user may determine the sensitivity of a model, for example the iDEA Absorption Module, to variations in permeability model, by varying the input permeability to the model. As an example using the iDEA Absorption Module, the input permeability was varied from 0.1 to 2.0 times the experimentally measured value while maintaining solubility at the experimentally measured value for the ten example drugs listed in Tables 10-14 of the “Example Data” chapter of the iDEA Manual, and incorporated herein by reference. For the iDEA Absorption Module, a two fold variation in the Caco-2 permeability resulted in an average difference of ±6 FDp units between the actual and predicted FDp and a maximum of +15 FDp units for the ten reference compounds.

By analyzing the ten reference compounds, and comparing the measured in vitro permeabilities with the in vitro data used in developing the permeability model, users can establish how their in vitro assays will affect the predictions from models using the output from the permeability model. For the iDEA Absorption Module example, if the measured CACO-2 permeability values fall with a two fold difference of the permeabilities reported for the ten reference compounds, then data from the users Caco-2 assay can be used-directly without affecting the predicted FDp in the IDEA Absorption Module. When the CACO-2 permeabilities fall outside this range, a correlation or map between the 10 reference permeabilities and the users CACO-2 permeabilities should be obtained. A linear or non-linear correlation may be used. The correlation selected typically will depend on the differences in the data. The selection and creation of a correlation function between the users data and the reference data is within the ordinary skill in the art of mathematical modeling. Two experiments are provided below to further illustrate this concept. Experiment One—Inter-Laboratory Correlation of Caco-2 Permeability

Purpose. To investigate inter-laboratory correlation of Caco-2 permeability using marker compounds. It is well known that in vitro permeability of drugs can vary lab-to-lab due to the difference of culture and transport conditions, but there is no standardized method to evaluate inter-laboratory variations.

### REFERENCES

1. Pade et al., Pharm. Res., 14: 1210(1997)/4.71 cm2, p35-41, age 15

2. Irvine et al., J. Pharm. Sci., 88: 28 (1999)/0.33 cm2, p31-42, age 21-25/MDCK: p59-80, age 3

3. Yamashita et al., EJPS., 10: 195 (2000)/4.71 cm2, age 18-21

4. Yazdanian et al., Pharm. Res., 15: 1490 (1998)/4.71 cm2, p 23-50, age-25

5. Yee., Pharm. Res., 14: 763 (1997)/0.83 cm2, p52-80, age 21

6. Rubas et al., Pharm. Res., 10: 113 (1993)/1.13.cm2,p30-35 age 20-26

7. Chong et al., Pharm. Res., 14: 1835 (1997)/4.19 cm2, p40-50, age 21-25/age 3

8. Liang et al., J. Pharm. Sci. 89: 336 (2000)/4.71 cm2, age 21-25/age 3-7

9. Withington et al., AAPS 1999. BD ViaSante

Methods. In vitro permeability of marker compounds representing various transport processes was studied in a Caco-2 monolayer (24-well transwell format/20-24 day culture/passage 30-40). The apical (A, 0.3 mL volume) and basolateral (B, 1.2 mL volume) reservoirs were filled with Ringer's buffer (pH 7.4, 290 mOsm/kg), containing 25 mM glucose. A<B permeability was studied at 37° C., 50 oscillations per minute (opm), 95% humidity, and 5% CO₂ using a 100 μM donor concentration with 1% dimethyl sufoxide (DMSO). Samples were collected over a 90 minute interval and aliquots analyzed by Liquid Chromatography/UV detection (LC/UV), Liquid Chromatography/Mass Spectrometry detection (LC/MS), or Liquid Scintillation Counting (LSC). In vitro permeability values of marker compounds were also collected from references with various culture (passage #, serum, filter type/size, seeding density, monolayer age) and various transport (agitation, pH, buffer) conditions, and they were correlated to the data used as a reference.

Results. The study results showed reproducible permeability for 4 transport markers at >4 individual Caco-2 monolayer batches (Mannitol: 0.461 (+0.074)×10⁻⁶ cm/s; atenolol: 1.07 (±0.43)×10⁻⁶ cm/s; propranolol: 28.7 (±6.22)×10⁻⁶ cm/s; and verapamil: 21.57 (±3.19)×10⁻⁶ cm/s). However, in vitro permeability values were variable 9 fold (0.38-3.23×10⁻⁶ cm/s) for mannitol, 45 fold (0.1-4.5×10⁻⁶ cm/s) for atenolol, 7 fold (14.8-110×10⁻⁶ cm/s) for propranolol, and 14 fold (3.83-51.9×10⁻⁶ cm/s) for verapamil in inter-laboratory Caco-2. It is also known that in vitro mannitol Apparent Permeability (Papp) values are variable 345 fold (0.019-6.55×10⁻⁶ cm/s) from published references. By contrast, the correlation of Caco-2 permeability between the data used as reference values and each of 10 references was good.

Conclusions. The in vitro permeability of marker compounds is reproducible under a standardized experimental condition, but can vary significantly between laboratories due to the difference of culture and experimental conditions. This work suggests that correlating inter-laboratory Caco-2 permeability may be a valuable standardization method to normalize inter-laboratory differences of Caco-2 permeability. Experiment Two—Effects of Various Experimental Conditions

Purpose. To investigate the effects of various experimental conditions on in vitro Caco-2 permeability using marker compounds.

Methods. In vitro permeability of various transport marker compounds was investigated using different apical pH's (pH 7.4 and pH 6.5), different buffers (Ringers, Mes, and Hepes), and different incubation (with or without CO₂) conditions. Caco-2 transepithelial transport studies were performed using a standardized 24-well transwell format (20-24 day culture/passage 30-40). The apical (A, 0.3 mL volume) and basolateral (B, 1.2 mL volume) reservoirs were filled with Ringer's buffer (pH 7.4, 290 mOsm/kg), containing 25 mM glucose. A→B and B→A permeability was studied at 37° C., 50 opm, 95% humidity, and 5% CO₂ using a 100 μM donor concentration with 1% DMSO. Monolayer integrity was evaluated by transepithelial electrical resistance (TEER) measurements. Samples were collected over a 90 minute interval and aliquots analyzed by LC/UV, LC/MS, or LSC.

Results. The change of apical pH from 7.4 to 6.5 increased the A to B Effective Permeability (Peff) for glycylsarcosine (PepT1 substrate, x6) and acidic compounds such as ibuprofen (×3) and ketoprofen (×6), but decreased the A to B Peff for basic compounds such as propranolol (×3), verapamil (×6), and vinblastine (×5). Conversely, the B to A Peff decreased in acidic compounds and increased in basic compounds. The efflux ratio was high for etoposide (8 at pH 7.4, 12 at pH 6.5) and vinblastine (4 at pH 7.4, 32 at pH 6.5) at apical pH of 7.4 and 6.5. The efflux ratios for atenolol, propranolol, and verapamil were high (>4) only at apical pH 6.5 probably due to the desorption effect at receiver compartment. The A→B and B→A Peff of marker compounds tested using Ringer's, Mes or Hepes buffer showed no change at apical pH 7.4, but differed at apical pH 6.5. When permeability studies were conducted at pH 7.4 Ringers in the absence of CO₂, the pH shifted to 7.8-8.0 during the study, so the resulting A to B Peff was decreased in glycylsarcosine (×2) and ketoprofen (×2). Additionally, when in vitro permeability values were collected from 10 references with various transport conditions, the permeability values were varied 45 fold (0.1-4.5×10⁻⁶ cm/s) for atenolol and 7 fold (14.8-110×10⁻⁶ cm/s) for propranolol.

Conclusions. Two different apical pH values, 6.5 and 7.4, showed pronounced effects on the permeability of both passively and actively transported marker compounds. This work suggests that the inter-laboratory differences of Caco-2 permeability can partially be explained by pH effect in different experimental conditions (apical pH, buffer, or CO₂ incubation). Experiment Three—Effect of Fasted State Simulated Intestinal Fluid

Purpose. To investigate the effect of fasted state simulated intestinal fluid (FaSSIF) on in vitro Caco-2 and rabbit intestinal permeability using marker compounds. FaSSIF is a physiological buffer containing bile salt and lecithin, and it has pronounced effects on the solubility (or dissolution) of class 11 compounds (BCS classification).

Methods. Twelve marker compounds representing higher solubility and various transport processes were used for in vitro permeability studies. Caco-2 transepithelial transport studies were performed using a standardized 24-well transwell format (20-24 day culture/passage 30-40). The transport experiments were performed using 0.3 mL apical solution either Ringers (pH 7.4), Mes (pH 6.5), or FaSSIF (pH 6.5, calcium free) and 1.2 mL basolateral solution (Ringer's buffer: pH 7.4, 290 mOsm/kg, 25 mM glucose). A<B and B→A permeability was studied at 37° C., 50 opm, 95% humidity, and 5% CO₂ using a 100 μM donor concentration with 1% DMSO. Monolayer integrity was evaluated by TEER measurements. Rabbit regional intestinal permeability studies were conducted using the Ussing side-by-side diffusion chamber, with volumes of 1.5 mL on each side. Intestinal segments (duodenum/jejunum/ileum/descending colon) were harvested from male New Zealand white rabbits (2.5-3.0 kg), stripped of the muscle layers and mounted in the chambers. Samples were collected over a 90 minute interval and aliquots analyzed by LC/UV, LC/MS, or LSC.

Results. FaSSIF increased the permeability of =paracellular markers in Caco-2, accompanied by a significant drop of TEER, possibly by the opening of tight junctions. When FaSSIF was used in the apical compartment and compared with Mes, the A→B permeability increased 3 fold (mannitol), 2 fold Polyethylene Glycol (PEG 4000), 5 fold (ribavirin), 3 fold (acyclovir), and 13 fold (ganciclovir), with the increase of B→A permeability by 11 fold (mannitol), 2 fold (PEG 4000), and 3 fold (ganciclovir). Addition of FaSSIF in the apical compartment also modulated actively transported markers in Caco-2. The A→B permeability increased 2 fold for etoposide (efflux), whereas decreased 2 fold for glycylsarcosine (influx). However, A→B and B→A permeability of transcellular markers was not changed by FaSSIF in Caco-2. Additionally, the permeability of either paracellular or transcellular markers was not changed by FaSSIF in rabbit intestine in either direction.

Conclusions. The results of this study suggest that the use of FaSSIF may open up tight junctions in Caco-2, leading to increased permeability of paracellular markers. This study also illustrates that FaSSIF may inhibit the active transporters (influx and efflux) in Caco-2. However, it needs further investigation for the effect of FaSSIF on in vivo permeability since there was inconsistency of FaSSIF effect on in vitro permeability between Caco-2 and rabbit intact intestine.

In summary, numerous benefits have been described which results from employing the concepts of the invention. The foregoing description of the presently preferred embodiments of the invention is presented for the purposes of illustration and description. It is not intended to be exhaustive or to limit the invention to a precise form disclosed. Obvious modifications or variations are possible in light of the above teachings. The embodiment was chosen to describe in order to best illustrate the principles of the invention and its practical application to thereby enable one of ordinary skill in the art to best utilize the invention in various embodiments and with various modifications as there suited particular use contemplated. It is intended that the scope of the invention be defined by the claims appended hereto.

What is claimed is:

1. A method for predicting permeability of a compound, the method comprising:
receiving as an input in vitro permeability and structure data for the compound; and
mapping the data into at least one permeability.

2. The method of claim 1 wherein mapping the data into at least one permeability comprises mapping the data into a plurality of permeabilities, each associated with a region in a mammalian GI tract.

3. The method of claim 2 wherein the mappings take into consideration solubility, permeability and at least one molecular descriptor associated with the compound.

4. The method of claim 3 further comprising:
adjusting the in vitro permeability data received to reduce errors caused by differences between test conditions under which the in vitro permeability data received was obtained and test conditions under which in vitro permeability data used to create the map was obtained.

5. A method for producing a model for predicting permeability of a compound, the method comprising:
receiving as an input in vitro permeability and structure data for a plurality of compounds;
obtaining for each compound at least one permeability; and
generating at least one model that associates the in vitro permeability and structure data with the related permeability.

6. The method of claim 5 wherein obtaining for each compound at least one permeability comprises obtaining a plurality of permeabilities, each permeability associated with a region in a mammalian GI tract.

7. The method of claim 5 or 6 wherein the model takes into consideration at least one of solubility or permeability and at least one molecular descriptor associated with the compound.

8. A permeability model, the model comprising:
means for receiving as an input in vitro permeability and structure data for a compound; and
means for converting the data into a permeability.

9. The model of claim 8 wherein the converting means converts the data into a plurality of permeability values, each value associated with a region in a mammalian GI tract.

10. The model of claim 9 wherein the data is converted into permeability values using one or more mappings.

11. The model of claim 10 wherein the map takes into consideration solubility, permeability and molecular descriptors associated with the compound.

12. A computer readable medium containing a regional intestinal permeability model, the medium comprising:
a computer readable medium; and
a data structure on the medium that converts in vitro permeability and structure data for a compound into a permeability value.

13. The medium of claim 12 wherein the data structure converts the data into a plurality of permeability values, each permeability value associated with a region in a mammalian GI tract.

14. The medium of claim 13 wherein the data is converted into permeability coefficients using mappings.

15. The medium of claim 14 wherein the mapping takes into consideration solubility, permeability and at least one molecular descriptor associated with the compound.

16. A method for producing a model for predicting permeability of a compound of interest, the method comprising:
obtaining in vitro permeability, structure, and permeability data for a plurality of compounds; and
developing a mapping between the in vitro permeability and compound structure data and the permeability data to create a model that predicts permeability.

17. The method of claim 16, wherein the model predicts a plurality of permeabilities, each permeability associated with a region in a mammalian GI tract for the compound of interest.
